(19) United States
(12) Patent Application Publication
(10) Pub. No.: US 2006/0223814 A1
(43) Pub. Date: Oct. 5, 2006

(54) Nk-2 antagonist basic linear compounds and formulations containing them

(76) Inventors: Daniela Fattori, Velletri (IT); Marina Porcelloni, Abbadia San Salvatore (IT); Pierro D'Andrea, Genzano (IT); Cristina Rossi, Romezia (IT); Maria Altamura, Firenze (IT); Carlo Alberto Maggi, Firence (IT)

Correspondence Address: Mark D. Lorusso;Lorusso Loud & Kelly LLP, Suite 312, 15 Rye Street, Portsmouth, NH, 03801, US

(21) Appl. No.: 10/554,152

(22) PCT Filed: Apr. 23, 2004
(86) PCT No.: PCT/EP04/50592

§ 371 Date: Oct. 22, 2005

(30) Foreign Application Priority Data
Apr. 24, 2003 (IT) FI2003A000113

(51) Int. Cl. A61K 31/496; A61K 31/453; A61K 31/381; C07D 409/02

(52) U.S. Cl. 514/252.13; 549/57; 544/376; 546/202; 514/320; 514/443

(57) ABSTRACT

The present invention describes compounds with formula (I) having linear structure basic properties useful as NK-2 antagonists; pharmaceutical compositions containing said compounds are also described and processes for their preparation.

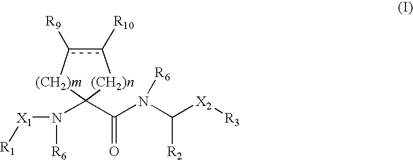

### FIELD OF THE INVENTION

The present invention relates to antagonists of tachykinins, in particular of neurokinin A, and to their use in pharmaceutical formulations.

In particular, the present invention relates to compounds with the following general formula:

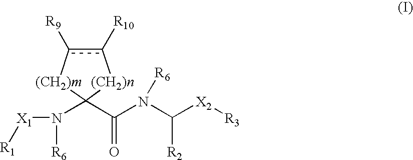

where:

X1 is a —NR6-CO—, —CO—, —NR6-CS— group

R1 is an aryl group selected from pyridine, thiophene, benzene, naphthalene, diphenyl, phenylthiophene, benzothiophene, benzofuran, N-indole by an R7 group, where said aryl group may also be substituted by one or more independent groups selected from halogen, C1-C6 alkyl optionally substituted by not more than three fluorine atoms (i.e. trifluoromethyl group), C1-C6 alkyloxyl, optionally substituted by not more than three fluorine atoms (i.e. trifluoromethyloxyl group), —OH, —NHR7, —N(R7)2, —SR7, —CONHR7, —COR7, —COOR7, —R8COOR7, —OR8COOR7, —R8COR7, —CONHR7, —R8CONHR7, —NHCOR7, -nitro, where R7 is hydrogen or C1-C6 alkyl with a linear or branched chain, and R8 is a C1-C6 alkylene group with a linear or branched chain;

R6 is selected from a group consisting of hydrogen or a C1-C6 alkyl with a linear or branched chain;

the broken line indicates a possible double bond and n and m may independently be 0, 1, 2;

R9 and R10 are selected independently in the hydrogen, C1-C6 alkyl group or may be connected to form an aromatic group selected in a phenyl group;

X2 is selected in the group formed of —(CH2)p-, —(CH2)q-CO—, —(CH2)s-O—(CH2)q-, —CH═CH—, —CH═CH—CO—, CH═CH—O—(CH2)q- where p may be 2, 3, 4; q may be 2, 3, 4: and s may be 1, 2;

R2 is selected from a group consisting of an aryl-alkyl or aryl radical where the aryl part is selected in a group consisting of benzothiophene, indole, pyridine, pyrrol, benzofuran, thiophene, benzene, naphthalene, imadazole, diphenyl, and may optionally be substituted by one or more substituents selected independently from halogen, C1-C6 alkyl optionally substituted by not more than three fluorine atoms (i.e. trifluoromethyl group), C1-C6 alkyloxyl, optionally substituted by not more than three fluorine atoms (i.e. trifluoromethyloxyl group), —OH, —NHR7, —N(R7)2, —SR7, —CONHR7, —COR7, —COOR7, —R8COOR7, —OR8COOR7, —R8COR7, —CONHR7, —R8CONHR7, —NHCOR7, -nitro, where R7 is hydrogen or C1-C6 alkyl with a linear or branched chain, and R8 is a C1-C6 alkylene group with a linear or branched chain;

R3 contains at least a basic amino group and is selected from a group with general formula:
—R₄—X₃—R₅

where R4 is selected from a group consisting of:
an —NR6- amino group;
an aliphatic heterocycle containing one or two heteroatoms selected from N, S and O, and optionally substituted by one or two C1-C6 alkyl groups;

X3 can be a simple bond or is selected in the group consisting of (CH2)t-, —CO—, —O—(CH2)t-, —O—, —NH—CO—CH2-, —NH—CO— where t can be 1, 2, 3;

R5 is:
an aliphatic heterocycle, selected in the group consisting of pyrrolidine, piperidine, morpholine, tetrahydropyran, 1,4-dioxa-8-azaspiro[4,5]decane, dioxane, optionally substituted by one or more C1-C6 alkyl, hydroxymethyl, —OH, cyanomethyl and C1-C6 alkyloxy groups;

a group selected from —NR₁₁R₁₂, —OR11 where R_{11,} R₁₂ are independently selected in the group: hydrogen, C1-C6 alkyl;
an aryl selected from thiophene, pyridine, furane or phenyl optionally substituted by one or more halogen, C1-C6 alkyl, C1-C6 alkyloxy and OH groups;

The present invention also includes “retro-inverted” compounds, that is, compounds having the structure of general formula (I), but wherein one or more amide bonds are reversed.

The presence of at least one amino group in R3, which imparts a basic characteristic to the compounds, may be considered an important structural characteristic.

The present invention also includes the pharmaceutically acceptable salts of compounds of formula (I) with organic and inorganic acids selected in the group: hydrochloric, sulphuric, phosphoric, acetic, trifluoroacetic, oxalic, malonic, maleic, fumaric, succinic, tartaric and citric acids. Moreover, all possible diastereoisomers or mixtures thereof, caused by introducing residues or groups having chiral centres into the sequence of general formula (I), are an integral part of the present invention.

The compounds of formula (I), with receptor antagonist activity of the tachykinins, prove useful to treat diseases wherein the neurokinin A plays a pathogenetic role.

### STATE OF THE ART

Tachykinins are a family of at least three peptides, known as Substance P, Neurokinin A (NKA) and Neurokinin B (NKB).

Research in the field of tachykinin antagonists, principally based on single or multiple substitution of the amino acids of the sequence of peptide agonists of Substance P and of the other tachykinins, has led to the discovery of nonapeptides containing one or more units of D-tryptophan (Regoli et al. Pharmacol 28,301 (1984)). However, the problems deriving from the pharmacological use of high molecular weight peptides (multiple sites of enzymatic hydrolytic attack, poor bio-availability, rapid hepatic and renal excretion) induced research of the minimum peptide fragment still capable of exerting antagonist activity. These studies led to the detection of adequately derivatized bicyclic and monocyclic peptides, antagonists of neurokinin A (Patent Applications WO9834949 and WO200129066).

Various compounds have been claimed as selective antagonists of Substance P, for example in WO9519966 and WO99845262, but, besides being selective for the NK1 receptor, these compounds have different structural characteristics to those of the present invention, the principal of which is the lack of a basic amino group.

Among NK1 antagonists, we can also mention those described in WO200014109; in these, there is no alpha, alpha-disubstituted amino acid, and the basic group, although present, is in very different positions with respect to the position of the compounds forming the object of the present invention.

Also in EP394989 the compounds with NK1 activity described do not generally have a basic group and do not have an alpha, alpha-disubstituted-amino acid. In Biorganic & Med. Chem. (1994), 2 (2), 101-113 (S. Boile et al.) compounds are described with NK2 antagonist activity containing an alpha, alpha-disubstituted phenylalanine, but they do not have basic characteristics and cannot be associated with the structure described with general formula I.

WO9404494 describes NK1 antagonists that have a alpha, alpha-disubstituted amino acid, but whose structure does not correspond to general formula (I), in particular for the presence, among others, of an —O—CO-group in place of X1.

### DETAILED DESCRIPTION OF THE INVENTION

It has been surprisingly found, and this is a characteristic of the present invention, that the compounds of general formula (I) as above defined, of a non-peptide nature, have improved characteristics in inhibiting bonding of tachykinins on the NK2 receptor and in vivo antagonist activity with respect to the products disclosed in the prior art patents cited above.

A preferred group of compounds of the present invention comprises the compounds that can be described by general formula (I) where the amino acid residue of general formula II:

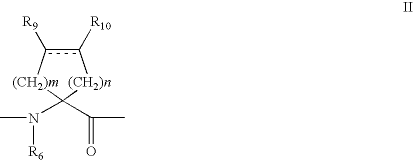

is selected in the group consisting of amino acid residues of: 1-aminocyclohexane-1-carboxylic acid (Ac6c), 1-aminocyclopentane-1-carboxylic acid (Ac5c), 1-aminocyclopent-3-ene-1-carboxylic acid (Ac5c), 1-aminoindane-1-carboxylic acid(1-Aic), 2-aminoindane-2-carboxylic acid (2-Aic), 2-aminotetraline-2-carboxylic acid (2-Atc), and the other groups are as defined above.

A group of preferred compounds according to the present invention consist of compounds having general formula (I), wherein:
X1 is a CO group

R1 is an aryl group selected from naphthalene, benzothiophene, benzofuran, N-indole substituted by an R7 group; where said aryl group is optionally substituted by one or more groups independently selected from halogen, C1-C6 alkyl optionally substituted by not more than three fluorine atoms (i.e. trifluoromethyl group), C1-C6 alkyloxy optionally substituted by not more than three fluorine atoms (i.e. trifluoromethoxyl group), —OH, —NHR7, —N(R7)2, —SR7, —CONHR7, —COR7, —COOR7, —R8COOR7, —OR8COOR7, —R8COR7, —CONHR7, —R8CONHR7, —NHCOR7, -nitro, where R7 is hydrogen or a linear or branched C1-C6 alkyl chain, and R8 is a linear or branched C1-C6 alkylene group;
R6 is selected from a group consisting of hydrogen or a linear or branched C1-C6 alkyl chain;
the amino acid residue of general formula II:

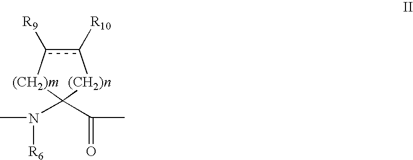

is selected in the group consisting of amino acid residues of: 1-aminocyclohexane-1-carboxylic acid (Ac6c), 1-aminocyclopentane-1-carboxylic acid (Ac5c),

R2 is a phenylmethyl group optionally substituted on the phenyl portion by one or two groups independently selected from halogen, C1-C6 alkyl, C1-C6 alkyloxy, and OH
X2 is as defined hereinbefore
R3 contains at least one basic amino group and represents a group:
   —R₄—X₃—R₅

wherein R4 is selected in the group:
an —NR6- amino group,
an aliphatic heterocycle selected from piperidine, piperazine, pyrrolidine optionally substituted by one or two C1-C6 alkyl groups;

X3 may be a simple bond or is selected in the group consisting of —(CH2)t-, —CO—, where t may be 1, 2, 3;

R5 is:
an aliphatic heterocycle selected in the group consisting of tetrahydropyran, morpholine, piperidine, optionally substituted by one or more groups C1-C6 alkyl, hydroxymethyl, —OH, cyanomethyl, and C1-C6 alkyloxy;
a group selected from —NR₁₁R₁₂, —OR11 where R_{11,} R₁₂ are independently selected in the group: hydrogen, C1-C6 alkyl;
an aryl selected from thiophene, furane or phenyl optionally substituted by one or more halogen, C1-C6 alkyl, C1-C6 alkyloxy or OH groups;

Particularly preferred amongst these are the compounds wherein:

XI is a —CO-group;

RI is a benzothiophene group, which may optionally be substituted by one or two groups selected independently from halogen, C1-C6 alkyl optionally substituted by not more than three fluorine atoms,

the amino acid residue of general formula (III) is 1-aminocyclopentane-1-carboxylic acid(Ac5c),

R6 is hydrogen;

R2 is phenyl-methyl, with the phenyl group optionally substituted by a C1-C6 alkyl;

X2 is selected in the group consisting of —(CH2)p-, —(CH2)q-CO—, —(CH2)s-O—(CH2)q-, —CH═CH—, —CH═CH—CO—, where p is 3; q is 2: and s is 1;

R3 contains at least one basic amino group and represents a group
—R₄—X₃—R₅

wherein

R4 is selected from a group consisting of:
an —NR6- amino group;
an aliphatic heterocycle selected from piperidine and piperazine

X3 may be a simple bond or is selected from the group consisting of —(CH2)t-, —CO—, where t may be 1, 2, 3;

R5 is:
a tetrahydropyran,
a group selected from —NR₁₁R₁₂, —OR11 where R_{11,} R₁₂ are independently selected in
a phenyl.

R6 is hydrogen;

Among the terms used in the present description the following are preferred: for halogens a group selected from fluorine, chlorine, bromine or iodine; for C1-C6 alkyl a group selected from methyl, ethyl, n-propyl, isopropyl, n-butyl, ter-butyl or, when optionally substituted by fluorine, trifluoromethyl; for C1-C6 alkyloxy a group wherein the alkyl part is selected from methyl, ethyl, n-propyl, isopropyl, n-butyl, ter-butyl or, when optionally substituted by fluorine, trifluoromethyl; for C1-C6 alkylene a group selected from methylene, ethylene, trimethylene, and tetramethylene.

The compounds of the present invention have shown an antagonist activity towards the action of Substance P, Neurokinin A, and Neurokinin B, although they proved particularly selective in antagonizing the action of Neurokinin A.

They may therefore be used as pharmaceuticals for the treatment and prevention of diseases in which tachykinins in general, and in particular Neurokinin A, are involved as neuromodulators.

Purely as an example, we can list respiratory diseases such as asthma, allergic rhinitis, and chronic obstructive bronchitis, ophthalmic diseases such as conjunctivitis, skin diseases such as allergic and contact dermatitis, and psoriasis, intestinal disorders such as irritable colon syndrome, ulcerous colitis and Crohn's disease, gastric diseases, urinary diseases such as cystitis and incontinence, erectile dysfunctions, diseases of the nervous central system such as anxiety, depression or schizophrenia, tumor diseases, autoimmune diseases or diseases related to AIDS, cardiovascular diseases, neuritis, neuralgia and treatment of pain, in particular visceral pain, inflammatory processes, such as osteoarthritis or rheumatoid arthritis.

The compounds of general formula (I), as defined above, can be prepared according to methods described in the literature and well known to those skilled in the art, such as amide condensation, substitution, addition or reductive amination reactions.

For example, these compounds can be synthesized by condensing the part of the molecule with basic characteristics, having the structure of formula III

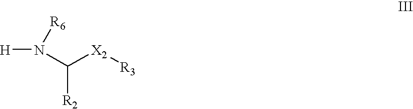

with the other part of the molecule, as an acid having general formula IV or as an oxazolidinone of formula V

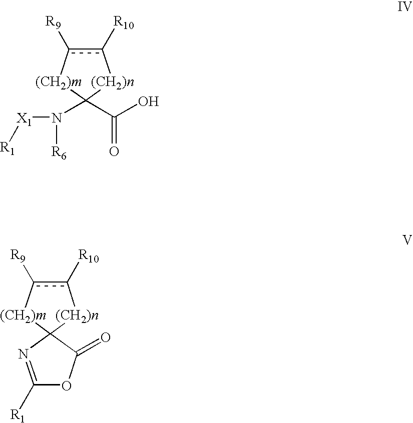

according to well known methods. In the part more specifically dedicated to the examples, various diagrams provide detailed descriptions of the synthetic paths followed for the various compounds described, although these synthetic paths and relative diagrams are provided purely as examples and must not be considered to be limiting.

The compounds of the present invention may exist in various isomeric forms. In fact, whereas the configuration of the carbon bonded to the substituent R₅ is univocally prefixed by using during synthesis the specific isomer of the amino acid derivative, frequently the other initial products can consist of mixtures of stereoisomers that are difficult separation. Therefore, the compounds of the present invention can be obtained as mixtures of diastereoisomers. These mixtures can be resolved by chromatography. The compounds of formula (1) can however be used both as single enantiomers and in the form of mixtures of isomers. Some representative examples of the present invention and of the method for the synthesis thereof are provided below.

### Synthesis Diagram for Compounds 6 and 7

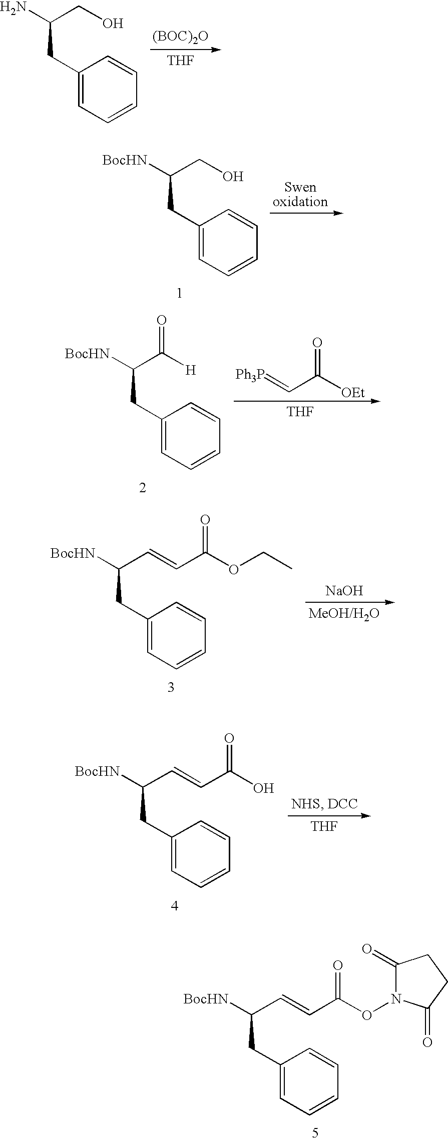

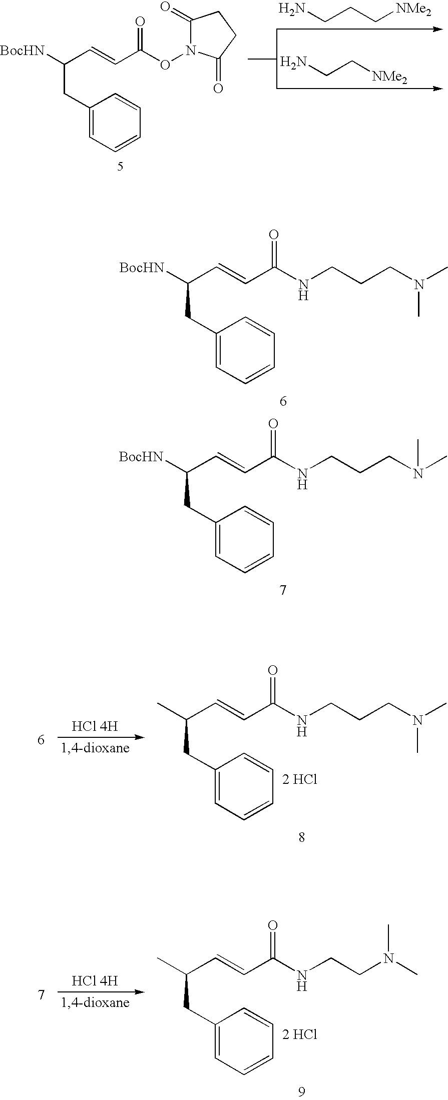

### EXAMPLE 1

### 1R-(1-Hydroxymethyl-2-phenyl-ethyl)-carbamic acid tert-butyl ester (1)

To a solution of D-phenyl alaninol (1.5 g, 9.9 mmol, 1 eq) in anhydrous THF (10 mL) under magnetic stirring is added di-t-butyl dicarbonate (2.7 mL, 11.9 mmol, 1.2 eq.) and then distilled under reduced pressure. The isolated residue is recrystallized from AcOEt/hexane to give 2.1 g (8.35 mmol, yield=84%) of (1-Hydroxymethyl-2-phenyl-ethyl)-carbamic acid tert-butyl ester (1). HPLC (method A): Rt=8.89 min.

H¹ NMR (δ, DMSO-d₆, 300 MHz): 1.31 (s, 9H, C(CH₃)₃); 2.54 (m, 1H, PhCHH); 2.81 (dd, 1H, PhCHH, J=5.4, 13.5 Hz); 3.15-3.40 (m, 2H, CH₂OH); 3.44-3.70 (m, 1H, OH); 4.63-4.77 (m, 1H, NHCH); 6.59 (d, 1H, NH, J=8.4 Hz), 7.07-7.14 (m, 5H, Ph).

### EXAMPLE 2

### 1R-(1-Formyl-2-phenyl-ethyl)-carbamic acid tert-butyl ester (2)

To a solution of oxalyl chloride distilled (1.5 mL, 11.94 mmol, 1.5 eq.) in DCM (20 mL) under magnetic stirring kept at −60° C. (a dry ice/acetone bath) under a nitrogen atmosphere is added anhydrous DMSO (1.70 mL, 23.87 mmol, 3.0 eq.) and the resulting mixture stirred at −60° C. for 10 minutes. A solution of (1-Hydroxymethyl-2-phenyl-ethyl)-carbamic acid tert-butyl ester (1) (2.0 g, 7.96 mmol, 1.0 eq.) in DCM (40 mL) at −60° C. is then added and the resulting mixture is kept under stirring for 15 minutes. Finally, DIPEA (8.15 mL, 47.75 mmol, 6.0 eq.) is added and the solution obtained kept at −60° C. for a further 5 minutes.

To the reaction left to heat to room temperature 60 mL of water are added and the resulting biphasic mixture is left under stirring for 10 minutes. The mixture is transferred to a separatory funnel, DCM (60 mL) is added, the mixture is shaken and the phases separated. The organic phase is then washed with a 1N solution of HCl (100 mL) and brine (100 mL), anhydrified on anhydrous Na₂SO₄, filtered and brought to dryness. (1-Formyl-2-phenyl-ethyl)-carbamic acid tert-butyl ester (2) is obtained as a pale yellow solid (1.98 g, 7.96 mmol, quantitative yield), cleaned by H¹-NMR which is used without further purification.

H¹ NMR (δ, DMSO-d₆, 300 MHz): 1.34 (s, 9H, C(CH₃)₃); 2.71 (dd, 1H, PhCHH, J=10.2, 13.8 Hz); 3.09 (dd, 1H, PhCHH, J=10.2, 13.8 Hz); 3.98-4.18 (m, 1H, NHCH); 7.12-7.44 ((m, 5H, Ph); 9.52 (s, 1H, COH).

### EXAMPLE 3

### 4R-4-tert-butoxycarbonylamino-5-phenyl-pent-2-enoic acid ethyl ester (3)

A mixture of (1-formyl-2-phenyl-ethyl)-carbamic acid tert-butyl ester (2) (1.98 g, 7.96 mmol, 1 eq.) and (carbethoxymethylene)triphenylphosphorane (2.93 g, 8.42 mmol, 1.06 eq.) in DCM (70 mL) is kept under magnetic stirring at room temperature for 3 hour. The solution is concentrated under reduced pressure and the raw product obtained is purified by flash chromatography eluting with a mixture of petroleum ether/ethyl acetate 90:100 until the first by-product (R_{f}=0.65 in petroleum ether: AcOet 90:10) is obtained and then with a mixture of petroleum ether: AcOEt 80:20. The fractions containing the desired product are recombined and the solvent distilled under reduced pressure. 4tert-butoxycarbonylamino-5-phenyl-pent-2-enoic acid ethyl ester (3) is obtained as a crystalline white solid (2.15 g, 6.73 mmol, yield=84.6%).

H¹ NMR (δ, DMSO-d₆, 300 MHz): 1.20 (t, 3H, CH₂CH₃, J=7.0 Hz); 1.31 (s, 9H, C(CH₃)₃); 2.71 (dd, 1H, PhCHH, J=9.3, 13.5 Hz); 2.85 (dd, 1H, PhCHH, J=5.4, 13.5 Hz); 4.11 (q, 2H, CH₂CH₃, J=7.0 Hz) 4.26-4.47 (m, 1H, NHCH); 5.82 (d, 1H, COCH, J=15.7 Hz); 6.85 (dd, 1H, COCHCH, J=5.4, 15.7 Hz); 7.09-7.42 (m, 5H, Ph).

### EXAMPLE 4

### 4R-4-tert-Butoxycarbonylamino-5-phenyl-pent-2-enoic acid (4)

4-tert-Butoxycarbonylamino-5-phenyl-pent-2-enoic acid ethyl ester (3) (2.15 g, 6.73 mmol) is hydrolyzed with NaOH 1N in MeOH/water. The reaction is immediate. HCl 2N is then added dropwise until total precipitation of the product which is collected by filtration on a Buckner funnel washing with water. 4-tert-Butoxycarbonylamino-5-phenyl-pent-2-enoic acid (4) is obtained as a white solid (1.75 g, 6.06 mmol, yield=90%). HPLC (method A): Rt=10.05 min.

H¹ NMR (δ, DMSO-d₆, 300 MHz): 1.30 (s, 9H, C(CH₃)₃); 2.71 (m, 1H, PhCHH); 2.83 (dd, 1H, PhCHH, J=6.0, 13.5 Hz); 3.97-4.43 (m, 1H, NHCH); 5.73 (d, 1H, COCH, J=15.7 Hz); 6.77 (dd, 1H, COCHCH, J=5.4, 15.7 Hz); 7.08-7.42 (m, 5H, Ph).

### EXAMPLE 5

### 4R-4-tert-Butoxycarbonylamino-5-phenyl-pent-2-enoic acid 2,5-dioxo-pyrrolidin-1-yl ester (5)

To a solution of 4-tert-Butoxycarbonylamino-5-phenyl-pent-2-enoic acid (4) (1.50 g, 5.14 mmol, 1.0 eq.) in THF (25 mL) kept at 0° C. using an ice bath are added N-hydroxysuccinimide (0.59 g, 5.14 mmol, 1.0 eq.) and subsequently N,N′-dicyclohexylcarbodiimide (1.06 g, 5.14 mmol, 1.0 eq.) in three parts in 10 minutes. The resulting mix is left under magnetic stirring at room temperature for 12 hours. The solid dicyclohexylurea formed is eliminated by filtration washing with THF. The solvent is then removed by distillation under reduced pressure to give 4-tert-Butoxycarbonylamino-5-phenyl-pent-2-enoic acid 2,5-dioxo-pyrrolidin-1-yl ester (5) as a spongy white solid (1.94 g, 5.00 mmol, yield=97%). HPLC (method A): Rt=10.56 min.

H¹ NMR (δ, DMSO-d₆, 300 MHz): 1.32 (s, 9H, C(CH₃)₃); 2.70-2.77 (m,1H, PhCHH); 2.83 (s, 4H, CH₂CH₂); 2.94 (dd,1H, PhCHH, J=5.3, 13.6 Hz); 4.32-4.71 (m, 1H, NHCH); 6.14 (d, 1H, COCH═CH, J=15.6 Hz); 7.06-7.49 (m, 5H, Ph+1H, COCH═CH).

### EXAMPLE 6

### 1R-[1-Benzyl-3-(3-dimethylamino-propylcarbamoyl)-allyl]-carbamic acid tert-butyl ester (6)

3-dimethylaminopropylamine (0.071 mL, 0.566 mmol, 1.1 eq.) is added to a solution of 4-tert-Butoxycarbonylamino-5-phenyl-pent-2-enoic acid 2,5-dioxo-pyrrolidin-1-yl ester (5) (0.200 g, 0.515 mmol, 1.0 eq.) in anhydrous THF (6.0 mL) and the resulting mixture left under magnetic stirring at room temperature for 3 hours. AcOEt (5 mL) and a 10% solution of NaHCO₃ (10 mL) are added and the phases separated. The organic phase is then washed with water (10 mL) and brine (10 mL), dried on Na₂SO₄, and then washed with filtered water and the solvent removed by evaporation under reduced pressure. The product [1-Benzyl-3-3-dimethylamino-propylcarbamoyl)-allyl]-carbamic acid tert-butyl ester (6) obtained is used in the subsequent reaction without further purification.

H¹ NMR (δ, DMSO-d₆, 300 MHz): 1.31 (s, 9H, C(CH₃)₃); 1.52 (t, 2H, NCH₂CH₂, J=7.0 Hz); 2.10 (s, 6H, N(CH₃)₂); 2.18 (t, 2H, NCH₂, J=7.2 Hz); 2.68-2.85 (m, 2H, CONCH₂); 2.95-3.19 (m, 2H, PhCH₂); 4.16-4.43 (m, 1H, NHCH); 5.88 (d, 1H, COCH, J=15.4 Hz); 6.56 (dd, 1H, COCHCH, J=5.7, 15.4 Hz); 7.07 (d, 1H, OCONH, J=8.7 Hz); 7.13-7.40 (m, 5H, Ph); 7.93-8.11 (m, 1H, NH).

Analogously, the following was prepared:

### EXAMPLE 7

### 1R-[1-Benzyl-3-(2-dimethylamino-ethylcarbamoyl)-allyl]-carbamic acid tert-butyl ester (7)

The product is used in subsequent reactions without further purification.

HPLC (method A): Rt=7.09 min.

### EXAMPLE 8

### 4R-4-Amino-5-phenyl-pent-2-enoic acid (3-dimethylamino-propyl)-amide (8)

[1-Benzyl-3-(3-dimethylamino-propylcarbamoyl)-allyl]-carbamic acid tert-butyl ester (6) is deprotected by treatment with a 4N solution of HCl in dioxane for 15 minutes at room temperature. The solvent is then removed by evaporation under reduced pressure. The solid obtained is triturated with Et₂O and isolated by filtration washing with Et₂O. 4-Amino-5-phenyl-pent-2-enoic acid (3-dimethylamino-propyl)-amide (8) is obtained as a clear yellow solid. HPLC (method A): Rt=3.89 min.

### EXAMPLE 9

4R-4-Amino-5-phenyl-pent-2-enoic acid (2-dimethylamino-ethyl)-amide (9) is obtained analogously starting from the derivative (7). HPLC (method A): Rt=3.55 min.

### Synthesis Diagram for Compounds 16, 17 and 19

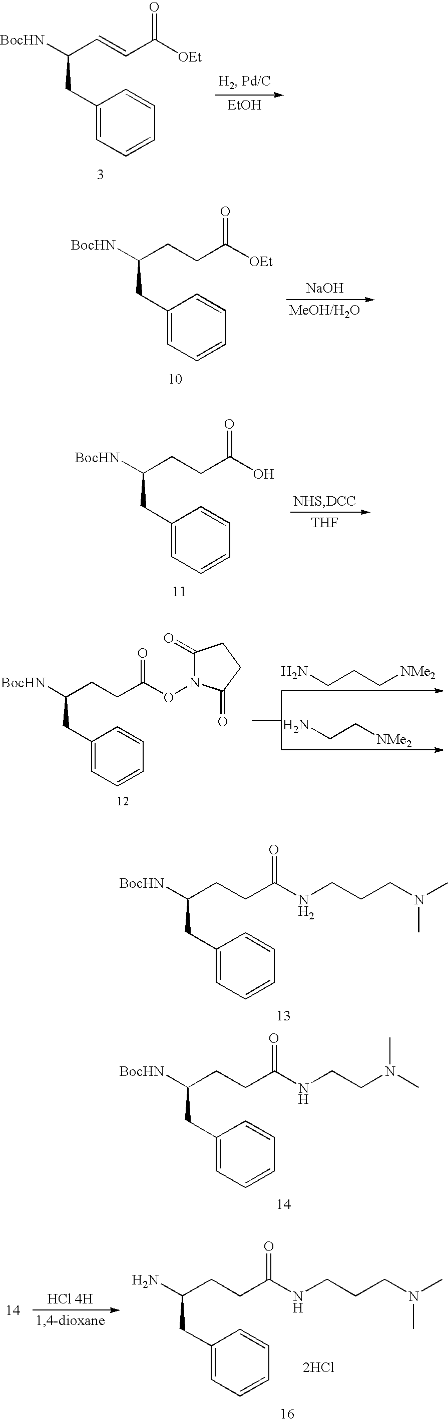

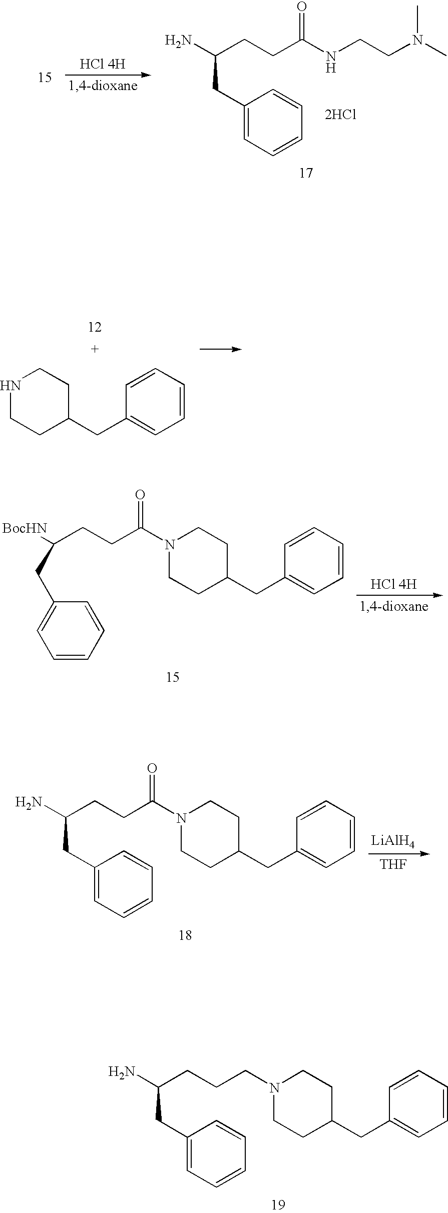

### EXAMPLE 10

### 4S-4-tert-Butoxycarbonylamino-5-phenyl-pentanoic acid ethyl ester (10)

4-tert-Butoxycarbonylamino-5-phenyl-pent-2-enoic acid ethyl ester (3) is reduced to the double bond by hydrogenation (static atmospheric pressure of H₂, Pd/C at 10%), according to a procedure known to those skilled in the art, to give 4-tert-Butoxycarbonylamino-5-phenyl-pentanoic acid ethyl ester (10) (2.10 g, 6.53 mmol) as white sold that is utilized in the subsequent reactions without further purification. HPLC (method A): Rt=11.66 min.

H¹ NMR (δ, DMSO-d₆, 300 MHz): 1.15 (t, 3H, CH₂CH₃, J=7.1 Hz); 1.32 (s, 9H, C(CH₃)₃); 1.44-1.60 (m, 1H, COCH₂CHH); 1.60-1.80 (m, 1H, COCH₂CHH); 2.18-2.40 (m, 2H, COCH₂); 2.60-2.74 (m, 2H, PhCH₂); 3.52-3.72 (m, 1H, NHCH); 4.00 (q, 2H, CH₂CH₃, J=7.1 Hz); 6.71 (d, 1H, NH, J=10.0 Hz); 7.11-7.40 (m, 5H, Ph).

### EXAMPLE 11

### 4S-4-tert-Butoxycarbonylamino-5-phenyl-pentanoic acid (11)

4-tert-Butoxycarbonylamino-5-phenyl-pentanoic acid ethyl ester (10) (2.00 g, 6.22 mmol) is hydrolyzed with NaOH 1N in MeOH/water. The reaction is immediate. HCl 2N is then added dropwise until total precipitation of the product which is collected by filtration on a Buckner funnel washing with water. 4-tert-Butoxycarbonylamino-5-phenyl-pentanoic acid (11) is obtained as a white solid (1.66 g, 5.66 mmol, yield=91%). HPLC (method A): Rt=9.28 min.

H¹ NMR (δ, DMSO-d₆, 300 MHz): 1.32 (s, 9H, C(CH₃)₃); 1.46-1.54 (m, 1H, COCH₂CHH); 1.62-1.70 (m, 1H, COCH₂CHH); 2.20 (m, 2H, COCH₂); 2.65 (m, 2H, PhCH₂); 3.60 (m, 1H, NHCH); 6.72 (d, 1H, NH, J=6.7 Hz); 7.16-7.29 (m, 5H, Ph); 11.99 (bs, 1H, OH);

### EXAMPLE 12

4S-8-tert-Butoxycarbonylamino-5-phenyl-pentanoic acid 2,5-dioxo-pyrrolidin-1-yl ester (12) is obtained by reaction of (11) (1.40 g, 4.77 mmol, 1.0 eq.) with DCC (1.08 g, 5.24 mmol, 1.1 eq) and NHS (0.55 g, 4.77 mmol, 1.0 eq.) following the procedure indicated in Example 5. The product is obtained as a white solid (1.53 g, 0.419 mmol, yield=88%).

HPLC (method A): Rt=10.39 min.

With an analogous procedure to the one described in Example 6 the compounds 13-15 are obtained by reaction of (12) with suitable amine.

### EXAMPLE 13

1S-[1-Benzyl-3-(3-dimethylamino-propylcarbamoyl)-propyl]-carbamic acid tert-butyl ester (13) is obtained as a very pale yellow oil. HPLC (method A): Rt=6.77 min.

H¹ NMR (δ, DMSO-d₆, 300 MHz): 1.32 (s, 9H, C(CH₃)₃); 1.45-1.75 (pseudo t+m, 2H+2H, NCH₂CH₂+COCH₂CH₂); 2.00-2.17 (m, 2H, COCH₂); 2.25 (s, 6H, N(CH₃)₂); 2.32-2.46 (m, 2H, (CH₃)₂NCH₂); 2.65 (d, 2H, CONCH₂, J=6.8 Hz); 3.03 (q, 2H, PhCH₂, J=6.2 Hz); 3.45-3.65 (m, 1H, NHCH); 6.70 (d, 1H, OCONH, J=8.8 Hz); 7.11-7.33 (m, 5H, Ph); 7.74-7.90 (m, 1H, NH).

### EXAMPLE 14

1S-[1-Benzyl-3-(2-dimethylamino-ethylcarbamoyl)-propyl]-carbamic acid tert-butyl ester (14) is obtained as a very pale yellow oil. HPLC (method A): Rt=6.45 min.

H¹ NMR (δ, DMSO-d₆, 300 MHz): 1.32 (s, 9H, C(CH₃)₃); 1.41-1.71 (m, 2H, COCH₂CH₂); 1.94-2.11 (m, 2H, COCH₂); 2.11 (s, 6H, N(CH₃)₂); 2.23 (t, 2H, J=6.8 Hz, (CH₃)₂NCH₂); 2.65 (d, 2H, CONCH₂, J=6.8 Hz); 3.09 (pseudo q, 2H, PhCH₂, J=6.4 Hz); 3.44-3.66 (m, 1H, NHCH); 6.68 (d, 1H, OCONH, J=8.7 Hz); 7.10-7.37 (m, 5H, ArH); 7.61-7.78 (m, 1H, NH).

### EXAMPLE 15

1S-[1-Benzyl-4-(4-benzyl)-piperidin-1-yl)-4-oxo-butyl]-carbamic acid tert-butyl ester (15) is obtained as a white solid from purification by flash chromatography using chloroform as eluent.

HPLC (method A): Rt=12.98 min.

H¹ NMR (δ, DMSO-d₆, 300 MHz): 0.90-1.12 (m, 2H, Hc); 1.32 (s, 9H, C(CH₃)₃); 1.38-1.65 (m, 2H, COCH₂CH₂); 1.56-1.85 (m, 2H+2H, COCH₂+Hd); 2.26 (m, 1H, Ha); 2.38(m, 1H, He); 2.48 (d, 2H, PhCH₂CHe, J=5.4 Hz); 2.66 (m, 2H, PhCH₂); 2.87 (t, 1H, Hb′, J=12.3 Hz); 3.59 (m, 1H, NHCH); 3.71-3.76 (m, 1H, Ha); 4.30-4.35 (m, 1H, Ha′); 6.72 (d, 1H, CONH, J=8.7 Hz); 7.16-7.20 (m, 5H, ArH); 7.22-7.40 (m, 5H, ArH).

With an analogous procedure to the one described in Example 8 the compounds 16 and 17 are obtained by deprotection reaction of 13 and 14.

### EXAMPLE 16

4S-4-Amino-5-phenyl-pentanoic acid (3-dimethylamino-propyl)-amide hydrochloride (16) which is used in subsequent reactions without further purification. HPLC (method A): Rt=3.69 min.

### EXAMPLE 17

4S-4-Amino-5-phenyl-pentanoic acid (2-dimethylamino-ethyl)-amide di-hydrochloride (17) which is used in subsequent reactions without further purification. HPLC (method A): Rt=3.45 min.

### EXAMPLE 18

4S-4-Amino-1-(4-benzyl-piperidin-1-yl)-5-phenyl-pentan-1-one hydrochloride (18) which is used in subsequent reactions without further purification.

MS (m/z): 351.5 (MH⁺). HPLC (method A): Rt=8.45 min

### EXAMPLE 19

### 1S-Benzyl-4-(4-benzyl-piperidin-1-yl)-butylamine (19)

The product described in the Example 18 (0.30 g, 0.77 mmol, 1.0 eq.) is reduced to the amide bond with LiAlH₄ (0.147 g, 3.88 mmol, 5.0 eq.) in anhydrous THF (10 mL) by reflux for one night. The reaction is quenched by cooling the flask in an ice bath and carefully adding first ice and then water. Et₂O (20 mL) and solid NaOH are then added in order to saturate the aqueous solution and the resulting biphasic mixture is stirred at room temperature for about 30 minutes. The various aluminium salts formed are eliminated by filtration on Celite® washing both with water and with ether. The solution obtained is then transferred to a separatory funnel and the phases separated. The organic phase is washed with water (20 mL) and brine (20 mL), dried on Na₂SO₄, filtered and the solvent removed by evaporation under reduced pressure. The oily product obtained is treated with HCl 4N in 1,4-dioxane to give 1-Benzyl-4-(4-benzyl-piperidin-1-yl)-butylamine (19) as pale yellow solid (0.23 g, 0.56 mmol, yield=73%) which is used in subsequent reactions without further purification.

MS (m/z): 337.8 (MH⁺). HPLC (method A): Rt=5.78 min.

### Synthesis Diagram for Compounds 27, 28 and 29

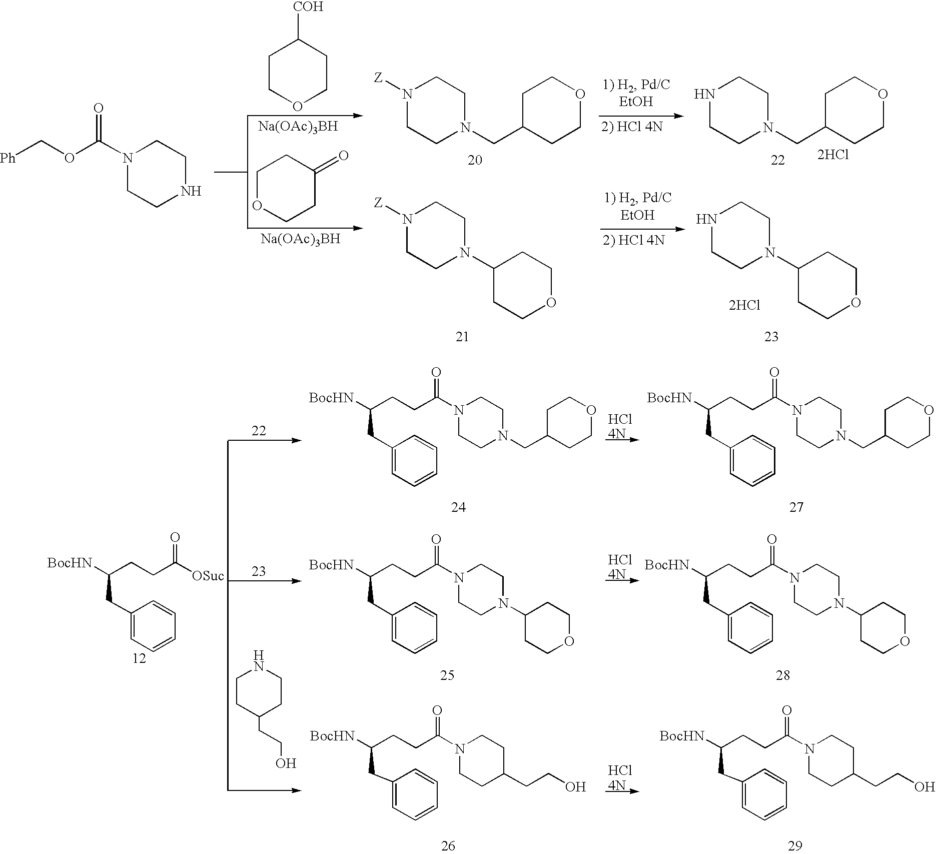

### EXAMPLE 20

### 4-(Tetrahydro-pyran-4-ylmethyl)-piperazine-1-carboxylic acid benzyl ester (20)

Piperazine-1-carboxylic acid benzyl ester (1.03 g, 4.68 mmol, 1.0 eq.) and 4-tetraidropiranil aldeide (0.8 mg, 7.0 mmol, 1.5 eq.) are dissolved in 20 ml of anhydrous DCM. Na(OAc)₃BH (1.48 g, 7.0 mmol, 1.5 eq.) are added to this opalescent solution. The reaction is left under magnetic stirring and under a nitrogen atmosphere at room temperature for 2 hours. When the reaction is completed the solvent is removed by evaporation under reduced pressure. AcOEt (20 mL) and a 1N solution of NaOH (20 mL) are added and the biphasic system transferred to a separatory funnel. The phases are separated and the organic phase is washed with water and brine, dried on Na₂SO₄, filtered and the solvent removed by evaporation under reduced pressure.

4-(Tetrahydro-pyran-4-ylmethyl)-piperazine-1-carboxylic acid benzyl ester (20) is obtained as a colourless oil (1.3 g, 4.08 mmol, yield=87%)

H¹ NMR (δ, DMSO-d₆, 300 MHz): 1.10 (qd, 2H, Hc, J=6.3, 13.5 Hz); 1.59 (d, 2H, Hd, J=12.6 Hz); 1.72 (m, 1H, He); 2.30 (m, 4H, Hh); 2.63 (m, 2H, Hf); 3.23-3.37 (m, 4H+2H, Hg+Hb), 3.80-3.84 (m, 2H, Ha), 5.07 (s, 2H, PhCH₂); 7.31-7.40 (m, 5H, ArH).

An analogous procedure to the one described in Example 20 is used to prepare compound 20 obtained as a colourless oil with a yield=85%.

### EXAMPLE 21

### 4-Tetrahydro-pyran-4-yl)piperazine-1-carboxylic acid benzyl ester (21)

HPLC (method A): Rt=5.68 min.

H¹ NMR (δ, DMSO-d₆, 300 MHz): 1.19 (qd, 2H, Hc, J=6.3, 13.5 Hz); 1.69 (d, 2H, Hd, J=12.6 Hz); 2.36-2.58 (m, 4H+1H, Hf+He); 3.15-3.53 (m, 4H+2H, Hg+Hb); 3.93 (m, 2H, Ha); 5.07 (s, 2H, PhCH₂); 7.27-7.45 (m, 5H, ArH).

### EXAMPLE 22

### 1-(Tetrahydro-pyran-4-ylmethyl)-piperazine di-hydrochloride

The product of Example 20 is deprotected by hydrogenation (H₂, Pd/C at 10%/o), according to a procedure known to those skilled in the art, to give 1-(Tetrahydro-pyran-4-ylmethyl)-piperazine which through treatment with HCl 4N in 1,4-dioxane and subsequent elimination of the solvent under reduced pressure produces the corresponding dichlorhydrate (22) as a white solid (1.03 g, 4.00 mmol, yield=98%).

H¹ NMR (δ, DMSO-d₆, 300 MHz): 1.12-1.32 (m, 2H, Hc); 1.70-1.80 (m, 2H, Hd); 2.03 (m, 1H, He); 3.06 (m, 2H, Hf); 3.29 (t, 2H, Hb, J=11.4 Hz); 3.40-3.80 (m, 4H+4H, Hg÷Hh); 3.81-3.87 (m, 2H, Ha); 9.6 (bs, 2H, ⁺NH₂); 11.2 (bs, 1H, ⁺NH)

### EXAMPLE 23

An analogous procedure to the one described in Example 22 is used to prepare compound 23 starting from 21

1-(Tetrahydro-pyran-4-yl)-piperazine di-hydrochloride (23) is obtained as a white solid (0.4 g, 1.64 mmol, yield=95%).

H¹ NMR (δ, DMSO-d₆, 300 MHz): 1.62-1.83 (m, 2H, Hc), 1.93-2.08 (m, 2H, Hd); 3.30 (t, 2H, Hb, J=11.4 Hz); 3.38-3.60 (m, 4H 4H, Hg+Hh); 3.61-3.81 (m, 1H, He); 3.95-4.03 (m, 2H, Ha); 9.6 (bs, 2H, ⁺NH₂); 12.0 (bs, 1H, ⁺NH).

### EXAMPLE 24

An analogous procedure to the one described in Example 6 is used to prepare compounds 24-26 starting from 11 and/or 22 or 23 or 4-piperidin ethanol.

1S-{1-Benzyl-4-oxo-4-[4-(tetrahydro-pyran-4-ylmethyl)-piperazin-1-yl]-butyl}-carbamic acid tert-butyl ester (24) is obtained as a yellow oil and used in the subsequent reactions without further purification.

HPLC (method A): Rt=7.21 min. MS (m/z): 460.3 (MH⁺).

### EXAMPLE 25

1S-{1-Benzyl-4-oxo-4-[4-(tetrahydro-pyran-4-yl)-piperazin-1-yl]-butyl}-carbamic acid tert-butyl ester (25) is obtained as a yellow oil and used in the subsequent reactions without further purification.

HPLC (method A): Rt=7.10 min. MS (m/z): 446.8 (MH⁺).

### EXAMPLE 26

1S-{1-Benzyl-4-[4-(2-hydroxy-ethyl)-piperidin-1-yl]-oxo-butyl}-carbamic acid tert-butyl ester (26) is obtained as a yellow oil and used in the subsequent reactions without further purification.

HPLC (method A): Rt=8.75 min.

An analogous procedure to the one described in Example 8 is used to prepare compounds 27-29 starting from 24-26

### EXAMPLE 27

4S-4-Amino-5-phenyl-1-[4-tetrahydro-pyran-4-ylmethyl)-piperazin-1-yl]-pentan-1-one di-hydrochloride (27) is obtained as a pale yellow solid and used in the subsequent reactions without further purification.

HPLC (method A): Rt=4.14 min. MS (m/z): 360.1 (MH⁺).

### EXAMPLE 28

4S-4-Amino-5-phenyl-1-[4-(tetrahydro-pyran-4-yl)-piperazin-1-yl]-pentan-1-one di-hydrochloride (28) is obtained as a pale yellow solid and used in the subsequent reactions without further purification.

HPLC (method A): Rt=4.06 min. MS (m/z): 346.9 (MH⁺).

### EXAMPLE 29

4S-4-Amino-1-[4-(2-hydroxy-ethyl)-piperidin-1-yl]-5-phenyl-pentan-1-one hydrochloride (29) is obtained as a white solid and used in the subsequent reactions without further purification.

HPLC (method A): Rt=5.06 min. MS (m/z): 304.9 (MH⁺).

An analogous procedure to the one described in Example 19 is used to prepare compounds 30 and 31 starting from 27 and 29.

### Synthesis Diagram for Compounds 30, 31 and 37

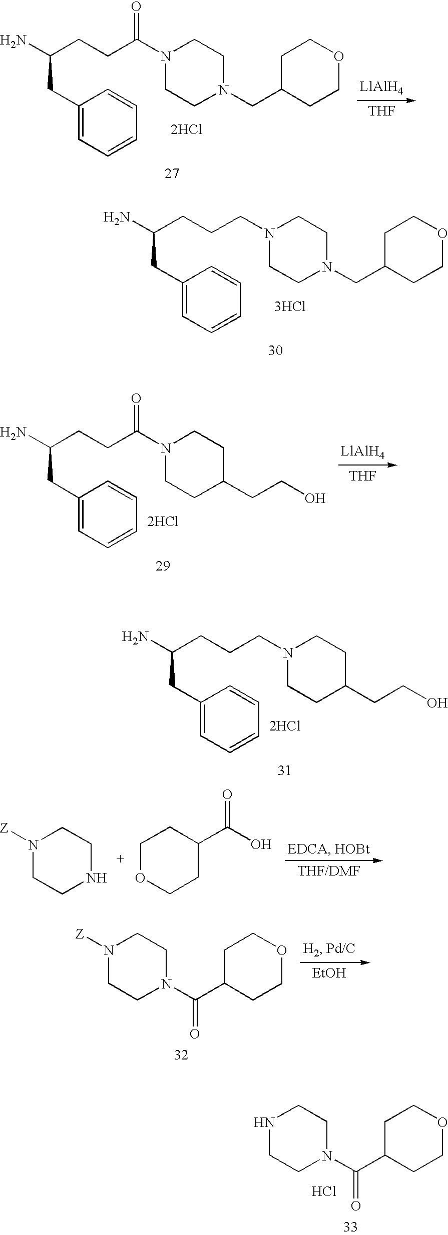

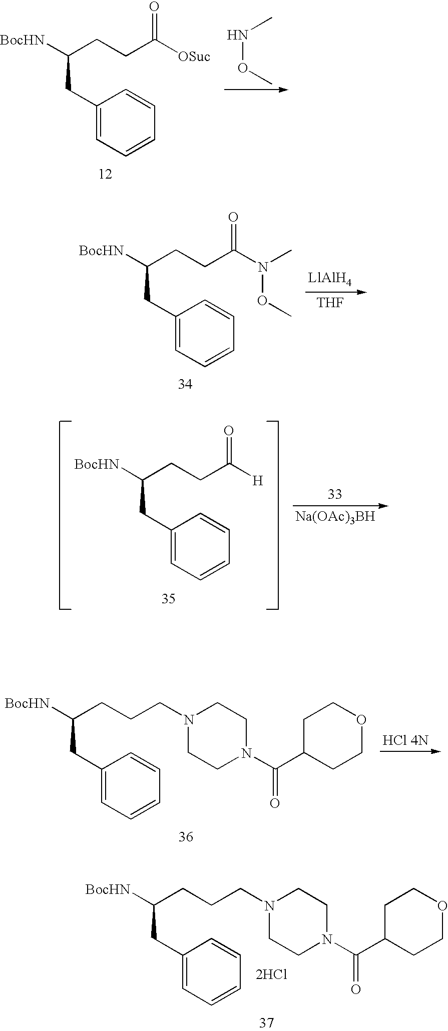

### EXAMPLE 30

1S-1-Benzyl-4-[4-(tetrahydro-pyran-4-ylmethyl)-piperazin-1-yl]-butylamine tri-hydrochloride (30) is obtained as a yellow oil and used in subsequent reactions without further purification. MS (m/z): 346.2 (MH⁺).

### EXAMPLE 31

2-[1-(4S-4-Amino-5-phenyl-pentyl)-piperidin-4-yl]-ethanol di-hydrochloride (31) is obtained as a yellow oil and used in subsequent reactions without further purification. MS (m/z): 291.1 (MH⁺).

### EXAMPLE 32

### 4-(Tetrahydro-pyran-4-carbonyl)-piperazine-1-carboxylic acid benzyl ester (32)

A mixture of tetrahydro-pyran-4-carboxylic acid (0.477 g, 2.27 mmol, 1.0 eq.) EDCA (0.480 g, 2.50 mmol, 1.1 eq.) and HOBt (0.340 g, 2.50 mmol, 1.1 eq.) in THF/DMF (8 mL/2 mL) is kept under magnetic stirring at room temperature for about 1 hour.

Piperazine-1-carboxylic acid benzyl ester (0.438 mL, 2.27 mmol, 1.0 eq.) is then added and the resulting reaction mixture is left to react for 14 hours. AcOEt (10 mL) and a 10% solution of NaHCO₃ (10 mL) are added and the phases are separated. The organic phase is then washed with water (2×10 mL) and brine (10 mL), dried on Na₂SO₄, filtered and the solvent removed by evaporation under reduced pressure. 4-(Tetrahydro-pyran-4-carbonyl)-piperazine-1-carboxylic acid benzyl ester (32) is obtained as an ivory coloured solid (0.613 g, 1.85 mmol, yield=81%) which does not require further prcation. HPLC (method A): Rt=7.71 min.

An analogous procedure to the one described in Example 22 is used to prepare compound 33 starting from 32.

### EXAMPLE 33

Piperazin-1-yl-(tetrahydro-pyran-4-yl)-methanone (33) is obtained as a colourless oil that does not require further purification. MS (m/z): 199.7 (MH⁺).

### EXAMPLE 34

### 1S-[1-Benzyl-3-(methoxy-methyl-carbamoyl)-propyl]-carbamic acid tert-butyl ester (34)

A solution of O,N-dimethyl-hydroxylamine hydrochloride (0.075 g, 0.77 mmol, 1.0 eq.) and DTPEA (0.131 mL, 0.77 mmol, 1.0 eq.) in DMF (2 mL) is added to a solution of 12 (0.300 g, 0.77 mmol, 1.0 eq.) in DMF (8 mL) and the resulting mixture is kept under magnetic stirring a 100° C. for one night. AcOEt (10 mL) and a 10% solution of NaHCO₃ (10 mL) are added and the phases are separated. The organic phase is then washed with water (2×10 mL) and brine (10 mL), dried on Na₂SO₄, filtered and the solvent removed by evaporation under reduced pressure. [1-Benzyl-3-(methoxy-methyl-carbamoyl)-propyl]-carbamic acid tert-butyl ester (34) is used in the subsequent reaction without further purification.

MS (m/z): 337.1 (MH⁺).

### EXAMPLE 35

### 1S-(1-Benzyl-4-oxo-butyl)-carbamic acid tert-butyl ester (35)

The product obtained from the previous reaction (34, Weinreb amide) (0.150 g, 0.446 mmol, 1.0 eq.) is reduced to aldehyde treating with LiAlH₄ (0.084 g, 2.230 mmol, 5.0 eq.) in THF (10 mL) at 4° C. for 15 minutes. Et₂O (5 mL) and a 5% aqueous solution of KHSO₄ are then added and the phases separated. The aqueous phase is washed with Et₂O (2×5 mL). The recombined organic phase is then washed with water (10 mL) and brine (10 mL), dried on Na₂SO₄, filtered and about half of the solvent removed by evaporation under reduced pressure. (1-Benzyl-4-oxo-butyl)-carbamic acid tert-butyl ester (35) is not isolated, but used as ether solution.

### EXAMPLE 36

### 1S-{1-Benzyl-4-[4-(tetrahydro-pyran-4-carbonyl)-piperazin-1-yl]-butyl}-carbamic acid tert-butyl ester (36)

Assuming a quantitative yield for the previous reaction, a reductive amination reaction is carried out between aldehyde 35 (0.446 mmol) as ether solution and amine 33 (0.044 g, 0.223 mmol, 0.5 eq.) in the presence of Na(OAc)₃BH (0.122 g, 0.580 mmol, 1.3 eq.) adding DCM (3 mL). The mixture obtained is left under magnetic stirring at room temperature for 14 hours. A 1N aqueous solution of NaOH (5 mL) is then added and the phases separated. The organic phase is then washed with water (10 mL) and brine (10 mL), dried on Na₂SO₄, filtered and the solvent removed by evaporation under reduced pressure. {1-Benzyl-4-[4-(tetrahydro-pyran-4-carbonyl)-piperazin-1-yl]-butyl}-carbamic acid tert-butyl ester (36) is obtained as a colourless oil that does not require further purification.

MS (m/z): 460.3 (MH⁺).

### EXAMPLE 37

An analogous procedure to the one described in Example 8 is used to prepare compound 37 starting from 36.

4S-[4-(4-Amino-5-phenyl-pentyl)-piperazin-1-yl]-(tetrahydro-pyranyl)-methanone di-hydrochloride (37) is obtained as a yellow solid and used in the subsequent reactions without further purification. MS (m/z): 360.1 (MH⁺).

### Synthesis Diagram for Compound 45

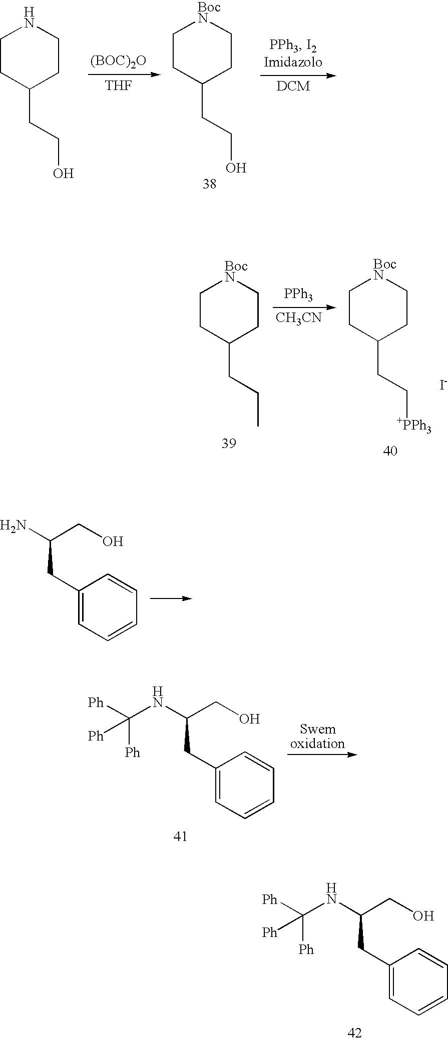

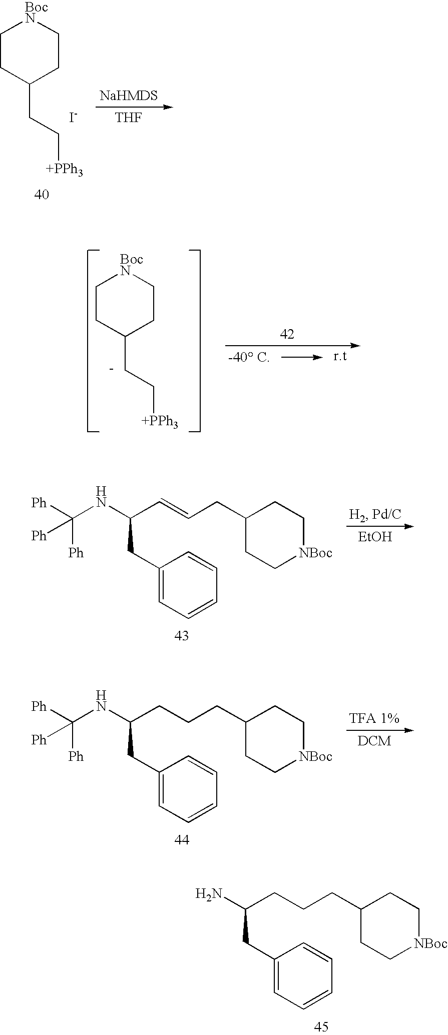

### EXAMPLE 38

### 4-(2-Hydroxy-ethyl)-piperidine-1-carboxylic acid tert-butyl ester (37)

A mixture of 4-piperidin ethanol (2.0 g, 15 mmol, 1.0 eq.) and t-butyl dicarbonate (3.87 mL, 18.5 mmol, 1.2 eq.) in anhydrous THF (80 mL) is kept under stirring at room temperature for one night. The solvent is then removed by evaporation under reduced pressure and the raw product obtained purified by flash chromatography using AcOEt: petroleum ether 60:40 as eluent mixture. 4-(2-Hydroxy-ethyl)-piperidine-1-carboxylic acid tert-butyl ester (37) is obtained as a colourless oil (3.1 g, 13.4 mmol, yield=87%)

HPLC (method A): Rt=7.92 min.

H¹ NMR (δ, CDCl₃-d₃, 300 MHz): 1.14 (dq, 2H, Hf, J=6.3, 13.5 Hz); 1.47 (s, 9H, C(CH₃)₃); 1.55 (pt, 2H, Hc, J=6.3 Hz); 1.50-1.65 (m, 1H, Hc); 1.69 (d, 2H, Hd, J=13.2 Hz); 1.89 (s, 1H, OH); 2.71 (td, 2H, Hb, J=2.1, 12.9 Hz); 3.73 (t, 2H, Hg, J=6.3 Hz); 4.10 (m, 2H, Ha).

### EXAMPLE 39

### 4-(2-Iodo-ethyl)-piperidine-1-carboxylic acid tert-butyl ester (39)

A solution of PPh₃ (1.20 g, 4.60 mmol, 1.3 eq.) and imidazole (0.32 g, 4.60 mmol, 1.3 eq.) temperature. A solution of 38 (0.81 g, 3.54 mmol, 1.0 eq.) in DCM (5 mL) is added and the resulting reaction mixture kept under stirring at room temperature for one night. Et₂O (100 mL) and water (50 mL) are added, the biphasic mixture transferred to a separatory funnel and the phases separated. The organic phase is subsequently washed with a saturated solution of NaHCO₃ (100 mL), a 5% aqueous solution of Na₂SO₃ (100 mL) and brine (100 mL), dried on Na₂SO₄, filtered and the solvent removed by evaporation under reduced pressure. The raw product obtained is purified by flash chromatography using petroleum ether. AcOEt 90:10 as eluent mixture. 4-(2-Iodo-ethyl)-piperidine-1-carboxylic acid tert-butyl ester 39 is obtained as a colourless oil (1.08 g, 3.19 mmol, yield=90%).

HPLC (method A): Rt=13.38 min.

H¹ NMR (δ, DMSO-d₆, 300 MHz): 1.04 (dq, 2H, Hf, J=4.3, 11.5 Hz); 1.39 (s, 9H, C(CH₃)₃); 1.46-1.56 (m, 1H, He); 1.62 (pd, 2H, Hc, J=14.1 Hz); 1.72 (q, 2H, Hd, J=6.9 Hz); 2.68 (m, 2H, Hg); 3.30 (m, 2H, Hb); 3.91 (m, 2H, Ha).

### EXAMPLE 40

### [2-(1-tert-Butoxycarbonyl-piperidin-4-yl)-ethyl]-triphenyl-phosphonium iodide (40)

A solution of 39 (0.696 mg, 2.05 mmol, 1.0 eq.) and PPh₃ (0.592 g, 2.259 mmol, 1.1 eq.) in CH₃CN (5 mL) is kept under stirring at the reflux temperature of the solvent for 2 days. The reaction mixture is then cooled and the solvent removed by evaporation under reduced pressure. The raw white solid obtained, consisting of the desired product and of the excess PPh₃ is left under stirring in Et₂O (10 mL) for 10 minutes. All the excess PPh₃ is solubilized whereas the white residue is collected by filtration washing with Et₂O. [2-(1-tert-Butoxycarbonyl-piperidin-4-yl)-ethyl]-triphenyl-phosphonium iodide (40) is obtained as a white solid (0.914 g, 1.52 mmol, yield=74%).

HPLC (method A): Rt=9.19 min.

H¹ NMR (δ,DMSO-d₆, 300 MHz): 0.96-1.10 (m, 2H, Hf); 1.38 (s, 9H, C(CH₃)₃); 1.45-1.60 (m, 2H+1H, Hc+He); 1.73 (d, 2H, Hd, J=12.0 Hz); 2.66 (m, 2H, Hg); 3.50-3.65 (m, 2H, Hb); 3.94 (m, 2H, Ha); 7.78-7.83 (m, 12H, ArH); 7.84-7.91 (m, 3H, ArH).

### EXAMPLE 42

### 2R-3-Phenyl-2-(trityl-amino)-propionaldehyde (42)

An analogous procedure to the one described in Example 2 is used to prepare compound 42 starting from 3-Phenyl-2-(trityl-amino)-propan-1-ol (41).

3-Phenyl-2-(trityl-amino)-propionaldehyde (42) is obtained as a very pale yellow solid (0.650 g, 1.66 mmol, yield=97%).

H¹ NMR (δ,DMSO-d₆, 300 MHz): 2.66 (m, 2H, PhCH₂); 3.18-3.25 (m, 1H, NHCHCO); 3.63 (d, 1H, NH, J=9.6 Hz); 7.15-7.34 (20 H, m, ArH); 8.72 (s, 1H, COH).

### EXAMPLE 43

### 4-[4R-5-Phenyl-4-(trityl-amino)-pent-2-enyl]-piperidine-1-carboxylic acid tert-butyl ester (43)

To a suspension of 40 (0.614 g, 1.02 mmol, 1.0 eq.) in anhydrous THF (3 mL) kept under stirring and under nitrogen atmosphere at room temperature is added NaHMDS 1M in THF (1 mL, 1.02 mmol, 1.0 eq.). After about 10 minutes the salt dissolves completely and the solution assumes a bright orange colour. The mixture is cooled to −40° C. and a solution of 42 (0.600 g, 1.53 mmol, 1.5 eq.) in anhydrous THF (3 mL) kept at −40° C. is added and the resulting reaction mixture kept under stirring under nitrogen atmosphere allowing the temperature to rise slowly to room temperature. Et₂O (10 mL) and brine (10 mL) are added and the phases separated. The organic phase is further washed with brine (10 mL) dried on Na₂SO₄, filtered and the solvent removed by evaporation under reduced pressure. The raw product obtained is purified by flash chromatography using as eluent mixture petroleum ether. AcOEt from 99:1 to 95:5. 4-[5-Phenyl-4-(trityl-amino)-pent-2-enyl]-piperidine-1-carboxylic acid tert-butyl ester (43) is obtained as a colourless oil (0.200 g, 0.34 mmol, yield=34%). HPLC (method A): Rt=11.65 min.

H¹ NMR (δ,DMSO-d₆, 300 MHz): 0.36-1.3 (m, 2H+5H, CH₂(Pip)+CH(CH₂)₂); 1.38 (s, 9H, C(CH₃)₃); 2.04-2.18 (m, 2H, PhCH₂); 2.41 (m, 2H, N(CHH)₂); 3.3 (m, 1H, NHCH); 3.72 (m, 2H, N(CHH)₂); 4.97 (m, 1H, NHCHCHCH); 5.29 (t, 1H, NHCHCH, J=10.4 Hz); 6.78 (d, 2H, ArH, J=7.2 Hz); 7.03-7.22 (m, 6H, ArH); 7.22-7.31 (m, 6H, ArH); 7.42-7.53 (m, 6H, ArH).

An analogous procedure to the one described in Example 10 is used to prepare compound 44 starting from 43.

### EXAMPLE 44

4-[4R-5-Phenyl-4-(trityl-amino)-pentyl]-piperidine-1-carboxylic acid tert-butyl ester (44) is obtained as a yellow oil and used in the subsequent reaction without further purification.

MS (m/z): 590.1 (MH⁺). HPLC (method A): Rt=11.68 min.

### EXAMPLE 45

### 4-(4R-4-Amino-5-phenyl-pentyl)-piperidine-1-carboxylic acid tert-butyl ester (45)

The raw reaction product 44 (0.340 g) is treated for 15 minutes with a 1% solution of TFA in DCM (5 mL). Two drops of water are then added and the solution left under stirring for a further 10 minutes. NaOH 2M (5 mL) is added and the phases separated. The aqueous phase is washed with DCM (2×10 mL) and the recombined organic phase dried on Na₂SO₄, filtered and the solvent removed by evaporation under reduced pressure. 4-(4-Amino-5-phenyl-pentyl)-piperidine-1-carboxylic acid tert-butyl ester (45) is obtained raw as a yellow oil and used in the subsequent reaction without further purification.

MS (m/z): 347.2 (MH⁺), 291 (-tBu), 247.2 (-BOC). HPLC (method B): Rt=3.96 min.

General Procedure for Coupling between Oxazolone and the Various Amines.

To a solution of 2-(benzo[b]thiophen-2-yl)-4-cyclopentyl-1,3-oxazolin-5-one (1.0 eq.) (Examples 46-50) or 2-(6-methyl-benzo[b]thiophen-2-yl)-4-cyclopentyl-1,3-oxazolin-5-one (1.0 eq.) (Examples 51-57) in DMF (10 mL) are added a solution of amine (1.0 eq.) and DIPEA (2.2 eq.) in DMF (3 mL) and the reaction mixture obtained is left under magnetic stirring at room temperature for 10 hours. AcOEt (10 mL) and a 10% aqueous solution of NaHCO₃ are added and the phases separated. The organic phase is then washed with brine (10 mL) dried on Na₂SO₄, filtered and the solvent removed by evaporation under reduced pressure. The raw product thus obtained is purified by flash chromatography using CHCl₃:MeOH 98:2 as eluent system. The following products were obtained with this procedure.

### EXAMPLE 46

### (R) Benzo[b]thiophene-2-carboxylic acid {1-[1-benzyl-3-(3-dimethylamino-propylcarbamoyl)-allylcarbamoyl]-cyclopentyl)-amide (46)

MS (m/z): 547.9 (MH⁺). HPLC (method A): Rt=7.78 min.

H¹ NMR (δ,DMSO-d₆, 300 MHz): 1.50-1.75 (m, 4H, cyclopen.); 1.87-2.07 (m, 2H+4H, CH₂CH₂CH₂+cyclopen.); 2.12 (s, 6H, N(CH₃)₂); 2.20 (m, 2H, NCH₂); 2.79 (m, 2H, PhCH₂); 3.10 (m, 2H, CONCH₂); 4.64 (m, 1H, NHCH); 5.86 (d, 1H, NHCOCH, J=15.4 Hz); 6.56 (dd, 1H, NHCHCH, J=5.8, 15.4 Hz); 7.06-7.20 (m, 5H, ArH); 7.46 (m, 1H+1H, C(6)H+C(5)H); 7.70 (m, 1H, CONHCH₂); 7.79 (m, 1H, NHCH); 7.91-8.07 (m, 1H+1H, C(4)H+C(7)H); 8.26 (s, 1H, C(3)H); 8.49 (s, 1H, NH-1Ac6c)

### EXAMPLE 47

### (R) Benzo[b]thiophene-2-carboxylic acid {1-[1-benzyl-3-(2-dimethylamino-ethylcarbamoyl)-allylcarbamoyl]-cyclopentyl}-amide (47)

MS (m/z): 533.85 (MH⁺). HPLC (method A): Rt=7.79 min.

H¹ NMR (δ,DMSO-d₆, 300 MHz): 1.50-1.75 (m, 4H, cyclopen.); 1.89-2.10 (m, 4H, cyclopen.); 2.13 (s, 6H, N(CH₃)₂); 2.27 (t, 2H, NCH₂, J=6.6 Hz); 2.80 (d, 2H, PhCH₂, J=7.2 Hz); 3.18 (m, 2H, CONCH₂); 4.57-4.75 (m, 1H, NHCH); 5.90 (d, 1H, NHCOCH, J=15.4 Hz); 6.57 (dd, 1H, NHCHCH, J=5.6, 15.4 Hz); 7.08-7.18 (m, 5H, ArH); 7.46 (m, 1H+1H, C(6)H+C(5)H); 7.62 (m, 1H, CONHCH₂); 7.70 (m, 1H, NHCH); 7.90-8.08 (m, 1H+1H, C(4)H+C(7)H); 8.26 (s, 1H, C(3)H); 8.49 (s, 1H, NH-1Ac6c).

### EXAMPLE 48

### (S) Benzo[b]thiophene-2-carboxylic acid {1-[1-benzyl-3-(3-dimethylamino-propylcarbamoyl)-propylcarbamoyl]-cyclopentyl}-amide (48)

MS (m/z): 549.2 (MH⁺). HPLC (method A): Rt=7.67 min.

H¹ NMR (δ,DMSO-d₆, 300 MHz): 1.51-1.75 (m, 2H+2H+4H, NCH₂CH₂+CH₂CH₂CO+cyclopen.); 1.85-2.05 (m, 2H+4H, COCH₂+cyclopen.); 2.07 (s, 6H, N(CH₃)₂); 2.14 (m, 2H, NCH₂CH₂); 2.63-2.72 (m, 2H, PhCH₂); 2.92-3.03 (m, 2H, CONCH₂); 3.91 (m, 1H, NHCH); 7.08-7.23 (m, 5H, ArH); 7.40 (m, 1H, NHCH); 7.46 (m, 1H+1H, C(6)H+C(5)H); 7.54 (m, 1H, CONHCH₂); 7.90-8.05 (m, 1H+1H, C(4)H+C(7)H); 8.26 (s, 1H, C(3)H); 8.52 (s, 1H, NH-1Ac6c)

### EXAMPLE 49

(S) Benzo[b]thiophene-2-carboxylic acid {1-[1-benzyl-3-(2-dimethylamino-ethylcarbamoyl)-propylcarbamoyl]-cyclopentyl}-amide (49) obtained as a trifluoroacetate salt through adding a solution of TFA in DCM and subsequent evaporation of the solvent under reduced pressure.

MS (m/z): 535.3 (MH⁺). HPLC (method A): Rt=7.95 min.

H¹ NMR (δ,DMSO-d₆, 300 MHz): 1.40-1.75 (m, 2H+4H, CH₂CH₂CO+cyclopen.); 1.83-2.07 (m, 2H+4H, COCH₂÷cyclopen.); 2.11 (s, 6H, N(CH₃)₂); 2.18-2.31 (m, 2H, (CH₃)₂NCH₂); 2.59-2.76 (m, 2H, PhCH₂); 2.98-3.12 (m, 2H, CONHCH₂); 3.83-3.91 (m, 1H, NHCH); 7.08-7.20 (m, 5H, ArH); 7.38 (m, 1H, NHCH); 7.40-7.50 (m, 1H+1H+1H, CONHCH₂+C(6)H+C(5)H); 7.90-8.07 (m, 1H÷1H, C(4)H+C(7)H); 8.25 (s, 1H, C(3)H); 8.51 (s, 1H, NH-1Ac6c).

### EXAMPLE 50

(S) Benzo[b]thiophene-2-carboxylic acid {1-[1-benzyl-4-(4-benzyl-piperidin-1-yl)-butylcarbamoyl]-cyclopentyl}-amide (50) MS (m/z): 608.3 (MH⁺).HPLC (method A): Rt=9.99 min.

H¹ NMR (δ,DMSO-d₆, 300 MHz): 1.20-2.10 (m, 9H+8H+2H+2H, pip.+cyclopen.÷NHCHCH₂CH₂); 2.71-3.09 (m, 2H+2H+2H, PhCH₂+PhCH₂+CH₂N); 3.83-4.17 (m, 1H. NHCH): 6.99-7.64 (m, 5H+5H+1H+1H+1H, ArH ArH+NHCH+C(6)H+C(5)H); 7.95 (m, 1H+1H, C(4)H+C(7)H); 8.26 (s, 1H, C(3)H); 8.31 (s, 1H, NH-1Ac6c).

### EXAMPLE 51

(S) 6-Methyl-benzo[b]thiophene-2-carboxylic acid (1-{1-benzyl-4-oxo-4-[4-tetrahydro-pyran-4-ylmethyl)-piperazin-1-yl]-butylcarbamoyl}-cyclopentyl)-amide (51) obtained as a trifluoroacetate salt through adding a solution of TFA in DCM and subsequent evaporation of the solvent under reduced pressure.

MS (m/z): 645.3 (MH⁺).HPLC (method A): Rt=8.22 min.

### EXAMPLE 52

### (S) 6-Methyl-benzo[b]thiophene-2-carboxylic acid (1-{1-benzyl-4-oxo-4-[4-(tetrahydro-pyran-4-yl)-piperazin-1-yl]-butylcarbamoyl}-cyclopentyl)amide (52)

MS (m/z): 631.2 (MH⁺). HPLC (method A): Rt=8.14 min.

H¹ NMR (δ,DMSO-d₆, 300 MHz): 1.18-1.34 (m, 2H, O(CH₂CHH)₂); 1.39-1.89 (m, 2H+8H, COCH₂CH₂+cyclopen.); 1.89-2.02 (m, 1H, COCHH); 2.02-2.13 (m, 1H, COCHH); 2.13-2.39 (m, 2H+4H+1H, O(CH₂CHH)₂+N(CH₂)₂+NCH₂CH2.44 (s, 3H, CH₃); 2.60-2.82 (m, 2H, PhCH₂); 3.16-3.40 (m, 2H+4H, O(CHH)₂+CON(CH₂)₂); 3.83 (dd, 2H, O(CHH)₂, J=3.0, 11.0 Hz); 3.88-4.00 (m, 1H, NHCH); 7.11-7.23 (m, 5H, ArH); 7.27 (d, 1H, C(5)H, J=7.4 Hz); 7.37 (d, 1H, NHCH, J=8.8 Hz); 7.74-7.86 (m, 1H+1H, C(4)H+C(7)H); 8.18 (s, 1H, C(3)H); 8.44 (s, 1H, NH-1Ac6c).

### EXAMPLE 53

### (S) 6-Methyl-benzo[b]thiophene-2-carboxylic acid (1-{1-benzyl-4-[4-(2-hydroxy-ethyl)-piperidin-1-yl]-4-oxo-butylcarbamoyl)-cyclopentyl)-amide (53)

MS (m/z): 576.2 (MH⁺). HPLC (method A): Rt=8.34 min

### EXAMPLE 54

### (S) 6-Methyl-benzo[b]thiophene-2-carboxylic acid (1-{1-benzyl-4-[4-(tetrahydro-pyran-4-ylmethyl)-piperazin-1-yl]-butylcarbamoyl}-cyclopentyl)-amide (54)

MS (m/z): 631.2 (MH⁺). HPLC (method A): Rt=7.48 min.

H¹ NMR (δ,DMSO-d₆, 300 MHz): 1.00-1.21 (m, 2H, O(CH₂CHH)₂); 1.22-1.46 (m, 5H); 1.51-1.67 (m, 6H); 1.83-2.08 (m, 6H); 2.08-2.25 (m, 8H+2H, Hpip+O(CH₂CHH)₂); 2.45 (s, 3H, CH₃); 2.61-2.73 (m, 2H, PhCH₂); 3.16-3.29 (m, 3H); 3.79-3.83 (m, 2H, O(CHH)₂; 3.94-4.00 (m, 1H, NHCH); 7.12-7.23 (m, 5H+1H, ArH+C(5)H); 7.28 (d, 1H, NHCH, J=8.4 Hz); 7.80-7.83 (m, 1H+1H, C(4)H+C(7)H); 8.15 (s, 1H, C(3)H); 8.34 (s, 1H, NH-1Ac6c).

### EXAMPLE 55

(S) 6-Methyl-benzo[b]thiophene-2-carboxylic acid (1-{1-benzyl-4-[4-(tetrahydro-pyran-4-carbonyl)-piperazin-1-yl]-butylcarbamoyl}-cyclopentyl)-amide (55) obtained as a trifluoroacetate salt through adding a solution of TFA in DCM and subsequent evaporation of the solvent under reduced pressure.

MS (m/z): 645.2 (MH⁺). HPLC (method A): Rt=7.48 min.

H¹ NMR (δ,DMSO-d₆, 300 MHz): 1.39-1.85 (m, 15H); 1.93 (m, 1H); 2.13 (m, 1H); 2.45 (s, 3H, CH₃); 2.45 (s, 3H, CH₃); 2.72 (d, 2H, PhCH₂, J=6.8 Hz); 3.00-3.14 (m, 6H); 3.38-3.43 (m, 5H); 3.83-4.43 (m, 2H÷H, O(CHH)₂+NHCH); 7.12-7.23 (m, 5H, ArH); 7.29 (d, 1H, C(5)H), J=8.0 Hz); 7.44 (d, 1H, NHCH, J=8.8 Hz); 7.78-7.85 (m, 1H+1H, C(4)H+C(7)H); 8.23 (s, 1H, C(3)H); 8.54 (s, 1H, NH-1Ac6c); 9.61 (bs, 1H, HN⁺).

### EXAMPLE 56

### (S) 4-[4-({1-[(6-Methyl-benzo[b]thiophene-2-carbonyl)-amino]-cyclopentane carbonyl}-amino)-5-phenyl-pentyl]-piperidine-1-carboxylic acid tert-butyl ester (56)

MS (m/z): 632.9 (MH⁺). HPLC (method A): Rt=8.89 min.

### EXAMPLE 57

An analogous procedure to the one described in Example 8 is used to prepare compound 57 starting from 56.

(S) 6-Methyl-benzo[b]thiophene-2-carboxylic acid [1-(1-benzyl-4-piperidin-4-yl-butylcarbamoyl)-cyclopentyl]-amide (57).MS (m/z): 532.8 (MH⁺).

### EXAMPLE 58

An analogous procedure to the one described in Example 20 is used to prepare compound 58 starting from 57 and 4-tetrahydropyranyl aldehyde.

(S) 6-Methyl-benzo[b]thiophene-2-carboxylic acid (1-{1-benzyl-4-[1-(tetrahydro-pyran-4-ylmethyl)-piperidin-4-yl]-butylcarbamoyl}-cyclopentyl)-amide (58) obtained as a trifluoroacetate salt through adding a solution of TFA in DCM and subsequent distillation of the solvent under reduced pressure.

MS (m/z): 630.3 (MH⁺). HPLC (method A): Rt=9.06 min.

### Synthesis Diagram for 62

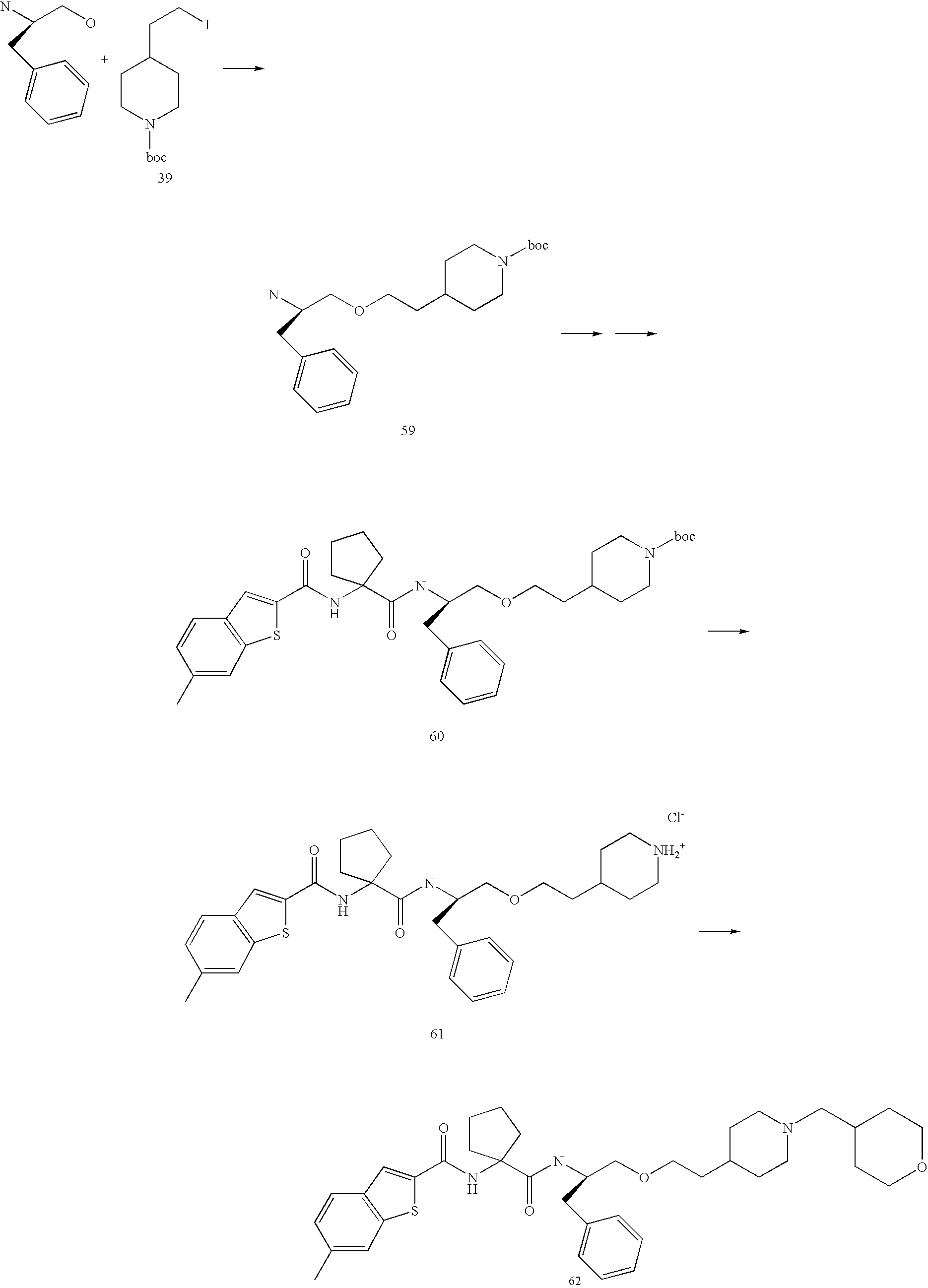

### EXAMPLE 59

### (R) 4-[2-(2-Amino-3-phenyl-propoxy)-ethyl]-piperidine-1-carbamic acid tert-butyl ester (59)

To a solution in anhydrous THF (100 mL), distilled on LiAlH₄, of D-phenyl alaninol (2.00 gr, PM=151, 13.24 mmol) is added potassium hydride (530 mg, PM=40, 13.2 mmol). The solution is left for two hours under stirring and under nitrogen at room temperature. The 4-(2-lodo-ethyl)-piperidine-1-carbamic acid tert-butyl ester 39 (4.5 gr, PM=339, 13.24 mmol), dissolved in 50 mL of anhydrous THF is then added through a dropping funnel. The reaction is left under stirring at room temperature for 12 hours. The solution is concentrated at reduced pressure, transferred to a separatory funnel with ethyl acetate and the organic phase is washed with NaOH 2N, brine and is dried on anhydrous Na₂SO₄. The raw reaction product is purified on a flash column (CHCl₃ 95/MeOH 5) obtaining 650 mg of 4-[2-(2-Amino-3-phenyl-propoxy)-ethyl]-piperidine-1-carbamic acid tert-butyl ester 59 (1.79 mmol, PM=362, Yield=14%)

MS (m/z): 263.1 (MH⁺) HPLC (method A): rt=8.14 min

¹H-NMR (δ, DMSO-d₆): 0.9-1.1 (dq,2H, CH₂); 1.4 (s,9H, (CH₃)₃); 1.45-1.7 (m, 5H, —CH—CH₂CH₂—O—, 2CH₂); 2.45(m, 1H, CH-D-phe-alaninol); 2.6-2.8 (m, 3H, —CH—N—CH—. —CH—D-phe); 3.0 (m, 1H, CH—NH₂); 3.1-3.5 (m, 4H, —CH₂—O—CH₂—); 3.8-3.9 (m, 2H, —CH—N—CH—); 7.1-7.3 (m,5H, Ar-D-phe-alaninol).

### EXAMPLE 60

### (R) 4-{2-[2-({1-[(6-Methyl-benzo[b]thiophene-2-carbonyl)-amino]-cyclopentanecarbonyl}-amino)-3-phenyl-propoxy]-ethyl}-piperidine-1-carbamic acid tert-butyl ester (60)

To a solution of 2-(6-Methyl-benzo[b]thiophene-2-il)-4-cyclopentyl-1,3-oxazolin-5-one (0.197 gr, PM 285, 0.69 mmol) in 10 mL of DMF is added 4-[2-(2-Amino-3-phenyl-propoxy)-ethyl]-piperidine-1-carbamic acid tert-butyl (0.25 gr, 0.69 mmol, PM=362) dissolved in 5 mL of DMF. The reaction is left at room temperature for 12 hours under stirring.

It is then transferred to a separatory funnel with ethyl acetate and the organic phase is washed with NaHCO₃ 10%, then with brine and dried on anhydrous Na₂SO₄. After evaporation of the solvent at reduced pressure the raw reaction product is chromalographed on a flash column (eluent chloroform:methanol 98/2).

0.40 g of 4{2[2({1[(6-Methylbenzo[b]thiophene-2-carbonyl)-amino]-cyclopentanecarbonyl}-amino)-3-phenyl-propoxy]-ethyl}-piperidine-1-carbamic acid tert-butyl ester 60 are obtained (yield 90%, PM=647, 0.62 mmol).

MS (m/z): 648.2 (MH⁺) HPLC(method A): rt=14.77

¹H-NMR (δ, DMSO-d₆): 0.9 (m,2H,CH₂);1.2-2.0 (m, 21H, 3CH₃, 6CH₂,); 2.15 (m,1H, CH—CH₂—CH₂—O); 2.45 (s,3H,CH₃); 2.6-2.8 (m, 4H, CH₂-D-phenylalaninol, CH—N—CH—); 3.2-3.4 (m, 4H, —CH₂—O—CH₂—); 3.85 (m, 2H, —CH—N—CH—); 4.05 (m, 1H, —CH—CH₂-Phe); 7.1-7.2 (m,5H, Ar-D-phenylalaninol), 7.25 (dd,1H, NH—CH D-phenylalaninol), 7.3 (d,1H,C(5)-H), 7.8 (s,1H,C(6)-H), 7.85 (d,1H,C(4)-H), 8.2 (s, 1H, C(3)-H), 8.45 (s, 1H,

### EXAMPLE 61

### (R) 6-Methyl-benzo[b]thiophene-2-carboxylic acid {1-[1-benzyl-2-(2-piperidin-4-yl-ethoxy)-ethylcarbamoyl]-cyclopentyl}-amide *Hydrochloride (61)

0.4 gr. of 4{2[2({1[(6Methylbenzo[b]thiophene-2-carbonyl)-amino]-cyclopentanecarbonyl}-amino)-3-phenyl-propoxy]-ethyl}-piperidine-1-carbamic acid tert-butyl ester (60, PM=647, 0.62 mmol) are dissolved in 5 mL or dioxane and 20 mL or a solution of HCl 4N in dioxane are added, under stirring and at room temperature; after 30 minutes the solution is evaporated and the gummy residue is dried twice by ethyl ether. The solid formed is triturated with ethyl ether and filtered on paper obtaining 0.33 g of 6-Methyl-benzo[b]thiophene-2-carboxylic acid {1-[1-benzyl-2-(2-piperidin-4-yl-ethoxy)-ethylcarbamoyl]-cyclopentyl}-amide *Hydrochloride 61 (PM=583.5, 91% yield, 0.56 mmol)

MS (m/z): 548.1 (MH⁺) HPLC(method A): rt=8.49 min

### EXAMPLE 62

### (R) 6-Methyl-benzo[b]thiophene-2-carboxylic acid [1-(1-benzyl-2-{2-[1-tetrahydro-pyran-4-ylmethyl)-piperidin-4-yl]-ethoxy}-ethylcarbamoyl)-cyclopentyl]-amide (62)

100 mg of 6-Methyl-benzo[b]thiophene-2-carboxylic acid {1-[1-benzyl-2-(2-piperidin-4-ylethoxy)ethylcarbamoyl]-cyclopentyl}-amide *Hydrochloride (61, PM=583.5, 0.17 mmol) and 58 mg of 4-tetrahydropyranyl aldehyde (PM=114, 0.51 mmol) are dissolved in 20 mL of anhydrous DCM. 110 mg of sodium triacetoxyborohydride (PM=212, 0.51 mmol) are added to this opalescent solution. The mixture is left under stirring and under nitrogen at room temperature for 2 hours. The solvent is evaporated at reduced pressure and extracted with ethyl acetate transferring the mixture to a separatory funnel; the organic phase is washed with NaHCO₃ brine and is placed to dry on Na₂SO₄. After evaporating the solvent at reduced pressure, the raw reaction product is purified on a flash column (eluent Chloroform:MeOH 95/5), obtaining 70 mg of 6-Methyl-benzo[b]thiophene-2-carboxylic acid [1-(1-benzyl-2-{2-[1-(tetrahydro-pyran-4-ylmethyl)-piperidin-4-yl]-ethoxy}-ethylcarbamoyl)-cyclopentyl]-amide 62 (PM=645, 0.108 mmol, 64% di yield)

MS (m/z): 646.1 (MH⁺) HPLC(method A): rt=9.01 min

¹H-NMR (δ, DMSO-d₆): 1.0-1.7 (m,18H,CH₂);1.85-2.0 (m,5H, CH—N—CH, CH—O—CH—tetrahydropyran, CH—CH₂—CH₂—N); 2.15 (m,1H, CH—CH₂—N); 2.45 (s,3H,CH₃); 2.65-2.8 (m, 4H, CH₂-D-phenyl-alaninol, CH—N—CH); 3.2-3.4 (m, 6H, —CH₂—O—CH₂—, —CH—O—CH-tetrahydropyran); 3.85 (m, 2H, —CH—O—CH— tetrahydropyran); 4.05 (m, 1H, —CH—CH₂— Phe); 7.1-7.2 (m,5H, Ar-D-phe-alaninol), 7.25 (dd,1H, NH—CH D-phe-alaninol), 7.3 (d,1H,C(5)-H), 7.8 (s,1H,C(6)-H), 7.85 (d,1H,C(4)-H), 8.2 (s, 1H, C(3)-H), 8.45 (s, 1H, CONH).

HPLC Methods:

Mobile phase: A=H₂O+0.1% TFA; B=MeCN+0.1% TFA

Method A

Column: Symmetry C18, 3.5 micron (4.6×100 mm)

Gradient: 1′ isocratic 10% B, 10′ of 10% B at 80% B

Flow velocity: 1 mL/min

λ=220, 254 nm.

Assessment of the antagonist activity on NK-2 receptors was performed with binding and functional tests according to prior descriptions in the literature for NK-2 antagonists.

In particular, affinity of the compounds for the human NK-2 receptor was assessed in a binding test using membranes of Chinese hamster ovary (CHO) cells, transfected with the NK-2 receptor of human ileum and the radioligand ['251]NKA (Amersham, aspecific activity 2000 Ci/mmol) at a concentration of 100 pM in competition studies. The substances under examination were tested in a concentration range from 0.01 nM to 10 mM. At the end of incubation (30 min., 20° C.) the samples were filtered and the radioactivity was determined using a gamma-counter.

The data in table 1 were obtained for some compounds of general formula (I) and concern the values of affinity to the human NK-2 receptor:

TABLE 1

| Compounds | pKi | Compounds | pKi |
|---|---|---|---|
| Example 46 | 9.2 | Example 50 | 9.9 |
| Example 51 | 10.1 | Example 52 | 10.1 |
| Example 53 | 8.7 | Example 54 | 10.3 |
| Example 55 | 10.0 | Example 62 | 9.3 |
| Example 58 | 9.9 | | |

The compounds of formula (I) can be handled according to the common pharmacopoeial techniques in order to prepare formulations suitable for oral, intranasal, parenteral, sublingual, inhalatory, transdermal, local or rectal use according to data known in the literature for this type of product; these forms of administration comprise oral formulations, such as tablets, capsules, powders, granulated formulations, and oral solutions or suspensions, formulations for sublingual administration, for intranasal administration for use in aerosol and implantation, formulations for subcutaneous, intramuscular, intravenous, intraocular and rectal administration. The effective doses are 0.1 to 50 mg/kg of body weight. For humans the dose may preferably range from 0.5 to 4000 mg/day, in particular from 2.5 to 1000 mg according to the patient's age and to the type of treatment. The treatment is carried out by administering the required amount to the patient 1 to 4 times per day for periods of up to 2 weeks or in any case until remission of symptoms; for chronic diseases, administration can be prolonged for significantly longer periods of time according to the judgment of the physician.

Thanks to their high antagonist activity on the NK-2 receptor of tachykinins, the present compounds are useful in the treatment of diseases in which Neurokinin A plays a pathogenetic role, and namely in the following diseases:
chronic obstructive respiratory diseases, such as asthma and allergic rhinitis, coughs and bronchitis;
opthalmic diseases, such as conjunctivitis or vitreoretinopathy;
skin problems, such as allergic and contact dermatitis, atopic dermatitis, eczema, itch, psoriasis, burns, in particular sunburn;
intestinal disorders, such as irritable colon, ulcerous colitis, Crohn's disease, diarrhoea;
gastric diseases, such as nausea or emesis;
prostatitis, neurological bladder, urinary incontinence, cystitis, urethritis, nephritis, erectile dysfunctions;
tumor diseases, autoimmune diseases or diseases associated with AIDS;
diseases of the central nervous system, such as anxiety, depression, schizophrenia, dementia, epilepsy, Parkinson's disease, Alzheimer's disease, drug and alcohol addiction, alcoholism, Huntington's chorea, neurodegenerative diseases and somatic disorders, such as stress;
treatment of pain, in particular visceralgia, neuritis, neuralgia;
cardiovascular diseases, such as hypertension, edema, thrombosis, angina, vascular spasms;
inflammatory diseases, such as arthritis, rheumatoid arthritis.

1) Compounds of general formula (I): 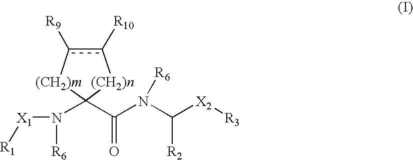
wherein:
X1 is a —NR6-CO—, —CO—, —NR6-CS— group;
R1 is an aryl group selected from pyridine, thiophene, benzene, naphthalene, diphenyl, phenylthiophene, benzothiophene, benzofuran, N-indole substituted by an R7 group, where said aryl group may also be substituted by one or more independent groups selected from halogen, C1-C6 alkyl optionally substituted by not more than three fluorine atoms (i.e. trifluoromethyl group), C1-C6 alkyloxyl, optionally substituted by not more than three fluorine atoms (i.e. trifluoromethyloxyl group), —OH, —NHR7, —N(R7)2, —SR7, —CONHR7, —COR7, —COOR7, —R8COOR7, —OR8COOR7, —R8COR7, —CONHR7, —R8CONHR7, —NHCOR7, -nitro, where R7 is hydrogen or C1-C6 alkyl with a linear or branched chain, and R8 is a C1-C6 alkylene group with a linear or branched chain;
R6 is selected from a group consisting of hydrogen or a C1-C6 alkyl with a linear or branched chain;
the broken line indicates a possible double bond and n and m may independently be 0, 1, 2;
R9 and R10 are selected independently in the hydrogen, C1-C6 alkyl group or may be connected to form an aromatic group selected in a phenyl group;
X2 is selected in the group formed of —(CH2)p-, —(CH2)q-CO—, —(CH2)s-O—(CH2)q-, —CH═CH—, —CH═CH—CO—, CH═CH—O—(CH2)q- where p may be 2, 3, 4; q may be 2, 3, 4: and s may be 1, 2;
R2 is selected from a group consisting of an aryl-alkyl or aryl radical where the aryl part is selected in a group consisting of benzothiophene, indole, pyridine, pyrrol, benzofuran, thiophene, benzene, naphthalene, imadazole, diphenyl, and may optionally be substituted by one or more substituents selected independently from halogen, C1-C6 alkyl optionally substituted by not more than three fluorine atoms (i.e. trifluoromethyl group), C1-C6 alkyloxyl, optionally substituted by not more than three fluorine atoms (i.e. trifluoromethyloxyl group), —OH, —NHR7, —N(R7)2, —SR7, —CONHR7, —COR7, —COOR7, —R8COOR7, —OR8COOR7, —R8COR7, —CONHR7, —R8CONHR7, —NHCOR7, —nitro, where R7 is hydrogen or C1-C6 alkyl with a linear or branched chain, and R8 is a C1-C6 alkylene group with a linear or branched chain;
R3 contains at least a basic amino group and is selected from a group with general formula:
—R₄—X₃—R₅
where R4 is selected from a group consisting of:
an —NR6- amino group;
an aliphatic heterocycle containing one or two heteroatoms selected from N, S and O, and optionally substituted by one or two C1-C6 alkyl groups;
X3 can be a simple bond or is selected in the group consisting of (CH2)t-, —CO—, —O—(CH2)t-, —O—, —NH—CO—CH2-, —NH—CO— where t can be 1, 2, 3;
R5 is:
an aliphatic heterocycle, selected in the group consisting of pyrrolidine, piperidine, morpholine, tetrahydropyran, 1,4-dioxa-8-azaspiro[4,5]decane, dioxane, optionally substituted by one or more C1-C6 alkyl, hydroxymethyl, —OH, cyanomethyl and C1-C6 alkyloxy groups;
a group selected from —NR₁₁R₁₂, —OR11 where R_{11,} R₁₂ are independently selected in the group: hydrogen, C1-C6 alkyl;
an aryl selected from thiophene, pyridine, furane or phenyl optionally substituted by one or more halogen, C1-C6 alkyl, C1-C6 alkyloxy and OH groups;
the pharmaceutically acceptable salts of compounds of formula (I) with organic and inorganic acids selected in the group: hydrochloric, sulphuric, phosphoric, acetic, trifluoroacetic, oxalic, malonic, maleic, fumaric, succinic, tartaric and citric acids; the possible optical isomers in the form of enantiomers or diastereoisomers, pure or in the form of racemic or non-racemic mixtures of said isomers; the “retro-inverted” compounds, that is, compounds having the structure of general formula (I), but wherein one or two amide bonds are reversed.

2) Compounds as claimed in claim 1, wherein the amino acid residue of general formula II: 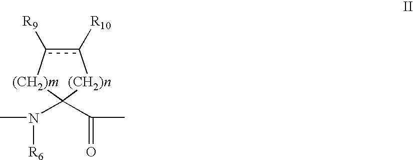
is selected in the group consisting of amino acid residues of: 1-aminocyclohexane-I-carboxylic acid, 1-aminocyclopentane-I-carboxylic acid, 1-aminocyclopent-3-ene-1-carboxylic acid, 1-aminoindane-I-carboxylic acid, 2-aminoindane-2-carboxylic acid, 2-aminotetraline-2-carboxylic acid,
and the other groups are as defined above.

3) Compounds as claimed in claim 2, wherein:
X1 is a CO group
R1 is an aryl group selected from naphthalene, benzothiophene, benzofuran, N-indole substituted by an R7 group; where said aryl group is optionally substituted by one or more groups independently selected from halogen, C1-C6 alkyl optionally substituted by not more than three fluorine atoms (i.e. trifluoromethyl group), C1-C6 alkyloxy optionally substituted by not more than three fluorine atoms (i.e. trifluoromethoxyl group), —OH, —NHR7, —N(R7)2, —SR7, —CONHR7, —COR7, —COOR7, —R8COOR7, —OR8COOR7, —R8COR7, —CONHR7, —R8CONHR7, —NHCOR7, -nitro, where R7 is hydrogen or a linear or branched C1-C6 alkyl chain, and R8 is a linear or branched C1-C6 alkylene group;
R6 is selected from a group consisting of hydrogen or a C1-C6 alkyl with a linear or branched chain;
the amino acid residue of general formula II: 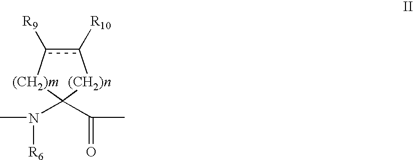
is selected in the group consisting of amino acid residues of: 1-aminocyclohexane-I-carboxylic acid, 1-aminocyclopentane-I-carboxylic acid,
R2 is a phenylmethyl group optionally substituted on the phenyl part by one or two groups independently selected from halogen, C1-C6 alkyl, CI-6 alkyloxy, and OH
X2 is as defined hereinbefore
R3 contains at least one basic amino group and represents a group:
—R₄—X₃—R₅
wherein R4 is selected in the group:
an —NR6- amino group,
an aliphatic heterocycle selected from piperidine, piperazine, pyrrolidine optionally substituted by one or two C1-C6 alkyl groups;
X3 may be a simple bond or is selected in the group consisting of —(CH2)t-, —CO—, where t may be 1, 2, 3;
R5 is:
an aliphatic heterocycle selected in the group consisting of tetrahydropyran, morpholine, piperidine, optionally substituted by one or more groups C1-C6 alkyl, hydroxymethyl, —OH, cyanomethyl, and C1-C6 alkyloxy;
a group selected from —NR₁₁R₁₂, —OR11 where R_{11,} R₁₂ are independently selected in the group: hydrogen, C1-C6 alkyl;
an aryl selected from thiophene, furane or phenyl optionally substituted by one or more halogen, C1-C6 alkyl, C1-C6 alkyloxy or OH groups.

4) Compounds as claimed in claim 3, wherein:
XI is a —CO-group;
RI is a benzothiophene group, which may optionally be substituted by one or two groups selected independently from halogen, CI-C6 alkyl optionally substituted by not more than three fluorine atoms,
the amino acid residue of general formula (III) is 1-aminocyclopentane-I-carboxylic acid,
R6 is hydrogen;
R2 is phenyl-methyl, with the phenyl group optionally substituted by a C1-C6 alkyl;
X2 is selected in the group consisting of —(CH2)p-, —(CH2)q-CO—, —(CH2)s-O—(CH2)q-, —CH═CH—, —CH═CH—CO—, where p is 3; q is 2: and s is 1;
R3 contains at least one basic amino group and represents a group:
—R₄—X₃—R₅
wherein
R4 is selected from a group consisting of:
an —NR6- amino group;
an aliphatic heterocycle selected from piperidine and piperazine
X3 may be a simple bond or is selected from the group consisting of —(CH2)t-, —CO—, where t may be 1, 2, 3;
R5 is:
a tetrahydropyran,
a group selected from —NR₁₁R₁₂, —OR11 where R_{11,} R₁₂ are independently selected in the group: hydrogen, methyl;
a phenyl.
R6 is hydrogen.

5) Compounds as claimed in claim 4, which are as follows:
(R) Benzo[b]thiophene-2-carboxylic acid {1-[1-benzyl-3-(3-dimethyl amino-propylcarbamoyl)-allylcarbamoyl]-cyclopentyl}-amide
(R) Benzo[b]thiophene-2-carboxylic acid {1-[1-benzyl-3-(2-dimethyl amino-ethylcarbamoyl)-allylcarbamoyl]-cyclopentyl}-amide
(S) Benzo[b]thiophene-2-carboxylic acid {1-[1-benzyl-3-(3-dimethyl amino-propylcarbamoyl)-propylcarbamoyl]-cyclopentyl}-amide
(S) Benzo[b]thiophene-2-carboxylic acid {1-[1-benzyl-3-(2-dimethyl amino-ethylcarbamoyl)-propylcarbamoyl]-cyclopentyl}-amide
(S) Benzo[b]thiophene-2-carboxylic acid {1-[1-benzyl-4-(4-benzyl-piperidin-1-yl)-butylcarbamoyl]-cyclopentyl}-amide
(S) 6-Methyl-benzo[b]thiophene-2-carboxylic acid (1-{1-benzyl-4-oxo-4-[4-(tetrahydro-pyran-4-ylmethyl)-piperazin-1-yl]-butylcarbamoyl}-cyclopentyl)-amide
(S) 6-Methyl-benzo[b]thiophene-2-carboxylic acid (1-{1-benzyl-4-oxo-4-[4-(tetrahydro-pyran-4-yl)-piperazin-1-yl]-butylcarbamoyl}-cyclopentyl)-amide
(S) 6-Methyl-benzo[b]thiophene-2-carboxylic acid (1-{1-benzyl-4-[4-(2-hydroxy-ethyl)-piperidin-1-yl]-4-oxo-butylcarbamoyl}-cyclopentyl)-amide
(S) 6-Methyl-benzo[b]thiophene-2-carboxylic acid (1-{1-benzyl-4-[4-(tetrahydro-pyran-4-ylmethyl)-piperazin-1-yl]-butylcarbamoyl}-cyclopentyl)-amide
(S) 6-Methyl-benzo[b]thiophene-2-carboxylic acid (1-{1-benzyl-4-[4-(tetrahydro-pyran-4-carbonyl)-piperazin-1-yl]-butylcarbamoyl}-cyclopentyl)-amide
(S) 6-Methyl-benzo[b]thiophene-2-carboxylic acid (1-{1-benzyl-4-[1-(tetrahydro-pyran-4-ylmethyl)-piperidin-4-yl]-butylcarbamoyl}-cyclopentyl)-amide
(R) 6-Methyl-benzo[b]thiophene-2-carboxylic acid [1-(1-benzyl-2-{2-[1-(tetrahydro-pyran-4-ylmethyl)-piperidin-4-yl]-ethoxy}-ethylcarbamoyl)-cyclopentyl]-amide.

6) Compounds as claimed in claim 1 for the preparation of pharmaceutical compositions useful in the treatment of diseases linked to stimulation of the NK-2 receptor.

7) Compounds as claimed in claim 6 for the preparation of pharmaceutical compositions for the treatment of respiratory diseases such as asthma, allergic rhinitis, ophthalmic diseases such as conjunctivitis, skin diseases such as allergic and contact dermatitis and psoriasis, intestinal disorders such as irritable colon syndrome, ulcerous colitis and Crohn's disease, urinary diseases such as cystitis and incontinence, erectile dysfunctions, diseases of the central nervous system such as anxiety, depression or schizophrenia, or tumor diseases, autoimmune diseases or diseases related to AIDS.

8) Pharmaceutical compositions containing as active ingredient at least one of the compounds of general formula (I) as claimed in claim 1, or mixtures thereof.

9) Pharmaceutical compositions as claimed in claim 8, also containing pharmaceutically acceptable excipients and diluents.

10) Pharmaceutical compositions as claimed in claim 8, for the treatment of diseases linked to stimulation of the NK-2 receptor and in particular for the treatment of respiratory diseases such as asthma and allergic rhinitis, opthalmic diseases such as conjunctivitis, skin diseases such as allergic and contact dermatitis and psoriasis, intestinal disorders such as irritable colon, ulcerous colitis and Crohn's disease, urinary diseases such as cystitis and incontinence, erectile dysfunctions, diseases of the central nervous system such as anxiety, depression and schizophrenia, or tumor diseases, autoimmune diseases or diseases related to AIDS.

11. Pharmaceutical compositions as claimed in claim 9, for the treatment of diseases linked to stimulation of the NK-2 receptor and in particular for the treatment of respiratory diseases such as asthma and allergic rinithis, ophtalmic diseases such as conjunctivitis, skin diseases such as allergic and contact dermatitis and psoriasis, intestinal disorders such as irritable colon, ulcerous colitis and Crohn's disease, urinary diseases such as cystitis and incontinence, erectile dysfunctions, diseases of the central nervous system such as anxiety, depression and schizophrenia, or tumor diseases, autoimmune diseases or diseases related to AIDS.
